(19) United States
(12) Patent Application Publication
(10) Pub. No.: US 2023/0215575 A1
(43) Pub. Date: Jul. 6, 2023

(54) SYSTEM AND METHOD FOR CHRONIC KIDNEY DISEASE

(71) Applicants: Richard BRADLEY, Melton Mowbray, Leicestershire (GB); MARS, INCORPORATED, McLean, VA (US)

(72) Inventors: Richard Bradley, Melton Mowbray, Leicestershire (GB); llias Tagkopoulos, Dixon, CA (US)

(73) Assignee: MARS, INCORPORATED, McLean, VA (US)

(21) Appl. No.: 18/000,406

(22) PCT Filed: Jun. 1, 2021
(86) PCT No.: PCT/US2021/035219

§ 371 Date: Dec. 1, 2022

Related U.S. Application Data
(60) Provisional application No. 63/038,552, filed on Jun. 12, 2020.
(60) Provisional application No. 63/033,154, filed on Jun. 1, 2020.

(51) Int. Cl. G16H 50/30; G16H 50/20; G16H 10/40

(52) U.S. Cl. CPC G16H 50/30; G16H 10/40; G16H 50/20

(57) ABSTRACT

The presently disclosed subject matter relates to methods or systems for identifying susceptibility of a dog to develop chronic kidney disease (CKD). The method, for example, can include receiving at least one of one or more biomarkers or demographic information of a dog. The method can also include processing at least one of the one or more biomarkers or demographic information of the dog using a prediction model. The prediction model can include a recurrent neural network. In addition, the method can determine a probability risk score of the dog for developing CKD based on the processed one or more biomarkers or demographic information.

### CROSS-REFERENCE TO RELATED APPLICATIONS

This application claims priority to U.S. Provisional Application No. 63/033,154, filed Jun. 1, 2020, and U.S. Provisional Application No. 63/038,552, filed Jun. 12, 2020, the contents of each of which are hereby incorporated by reference in their entireties.

### TECHNICAL FIELD

The presently disclosed subject matter relates to methods and systems for determining a pet’s susceptibility to developing chronic kidney disease (CKD).

### BACKGROUND

Chronic kidney disease (CKD), also known as chronic renal disease or chronic renal failure, is a progressive loss in renal function over a period of months or years. CKD can be caused by a variety of conditions and mechanisms, and it affects both humans and pets. The incidence of dog or canine CKD has been estimated to be around 0.5-1.0% of dogs in the United States but has been shown to approach 25% in some populations, including certain breeds with known predisposition. Dog or canine CKD is also generally considered to have a worse prognosis and shorter survival times compared to other pets, such as felines.

Given the increase risk associated with dog or canine CKD, there remains a need for systems and methods that can help with early detection or diagnosis of CKD. There further remains a need for providing a customized recommendation to help to reduce the health risks associated with CKD.

### SUMMARY

In certain non-limiting embodiments, the presently disclosed subject matter provides a computer system for identifying susceptibility of a dog to develop chronic kidney disease (CKD). The computer system can include a processor and a memory that stores code that, when executed by the processor, cause the computer system to receive at least one of one or more biomarkers of the dog, where the one or more biomarkers can include information relating to at least one of a urine specific gravity, a creatinine, a urine protein, a blood urea nitrogen (BUN), or demographic information of the dog, where the demographic information can include at least one of age or weight of the dog. The computer system can also be caused to process at least process at least one of the one or more biomarkers or demographic information of the dog using a prediction model. The prediction model can include a recurrent neural network. In addition, the computer system can be caused to determine a probability risk score of the dog for developing CKD based on the processed one or more biomarkers. In certain other non-limiting embodiments, the one or more biomarkers can include information relating to an amylase.

In certain non-limiting embodiments, the computer system can be caused to determine a customized recommendation based on the probability risk of the dog for developing CKD. The customized recommendation can include at least one of one or more therapeutic interventions, one or more dietary recommendations, one or more renal sparing strategies, or one or more tests for disease progression. The dietary recommendation can include the recommended use or the use of one or more pet products, such as a pet food product, and/or the recommended use or the use of any combination of pet products. Moreover, the dietary recommendation can include a recommendation of a dietary change, the recommendation of a dietary regimen, and/or a recommendation of a supplement, such as a dietary supplement or a pharmaceutical supplement, for a dog. In another example, the one or more renal sparing strategies can include avoidance of non-steroidal anti-inflammatories, aminoglycosides, or any combination thereof, and/or the one or more tests for disease progression can include testing of serum parathyroid hormone levels. In some non-limiting embodiments, the customized recommendations can be transmitted to a user equipment of a veterinarian, owner, or caregiver of the dog.

In certain non-limiting embodiments, the recurrent neural network can include a hidden layer architecture with three layers. The three layers can include a first layer with five nodes, a second layer with three nodes, and a third layer with three nodes. The recurrent neural network can undergo a ten-fold cross-validation process and/or can be trained over eight or eighteen epochs. The decision threshold for developing the CKD using the recurrent neural network can be about 0.5. In another example, the decision threshold for developing the CKD using the recurrent neural network can be about 0.3 to about 0.9. In yet another example, the decision threshold can be between about 0 to about 1. In some non-limiting embodiments, the recurrent neural network can be trained using a training dataset. The training dataset can include the one or more biomarkers and the demographic information for a plurality of other dogs. In certain non-limiting embodiments, the prediction model can further include the recurrent neural network with long short-term memory (LSTM).

In certain non-limiting embodiments, the presently disclosed subject matter provides a method for identifying susceptibility of a dog to develop CKD. The method can include receiving at least one of one or more biomarkers of a dog, where the one or more biomarkers include information relating to at least one of a urine specific gravity, a creatinine, a urine protein, a BUN, or demographic information of the dog, where the demographic information can include at least one of age or weight of the dog. The method can also include processing at least one of the one or more biomarkers or demographic information of the dog using a prediction model. The prediction model can include a recurrent neural network. In addition, the method can include determining a probability risk score of the dog for developing CKD based on the processed one or more biomarkers.

In certain non-limiting embodiments, the presently disclosed subject matter provides a computer system for identifying susceptibility of a dog to develop CKD. The computer system includes a processor and a memory that stores code that, when executed by the processor, causes the computer system to receive at least one of one or more biomarkers of a dog, where the one or more biomarkers can include information relating to at least one of a urine specific gravity, a creatinine, a urine protein, a BUN, or demographic information of the dog, where the demographic information can include at least one of age or weight of the dog. The computer system can also be caused to process at least one of the one or more biomarkers or demographic information of the dog using a prediction model. The prediction model can include a recurrent neural network. The recurrent neural network includes a hidden layer architecture with three layers, the three layers comprising a first layer with five nodes, a second layer with three nodes, and a third layer with three nodes. In addition, the computer system can also be caused to determine a probability risk score of the dog for developing CKD based on the processed one or more biomarkers.

In certain non-limiting embodiments, the presently disclosed subject matter provides a computer system for identifying susceptibility of a dog to develop CKD. The method can include receiving at least one of one or more biomarkers of a dog, where the one or more biomarkers can include information relating to at least one of a urine specific gravity, a creatinine, a urine protein, a BUN, or demographic information of the dog, where the demographic information can include at least one of age or weight of the dog. The method can include processing at least one of the one or more biomarkers or demographic information of the dog using a prediction model. The prediction model can include a recurrent neural network. The recurrent neural network includes a hidden layer architecture with three layers, the three layers comprising a first layer with five nodes, a second layer with three nodes, and a third layer with three nodes. In addition, the method can include determining a probability risk score of the dog for developing CKD based on the processed one or more biomarkers.

### BRIEF DESCRIPTION OF THE DRAWINGS

FIGS. 1A - 1H illustrate distribution charts of the study data set according to certain embodiments described herein;

FIGS. 2A - 2L illustrate example electronic health records (EHRs) with no CKD according to certain embodiments described herein;

FIGS. 3A - 3L illustrate example electronic health records (EHRs) with CKD according to certain embodiments described herein;

FIG. 4 illustrates a computer system according to certain embodiments described herein;

FIG. 5 illustrates a more detailed view of a server of FIG. XX, according to certain embodiments described herein;

FIG. 6 illustrates a user equipment according to certain embodiments described herein;

FIG. 7 illustrates model performance as a function of age according to certain embodiments described herein;

FIG. 8 illustrates model sensitivity as a function of the number of visits according to certain embodiments described herein;

FIG. 9 illustrates model sensitivity as a function of time before diagnosis according to certain embodiments described herein;

FIGS. 10A - 10E illustrate pre-processing of the dataset according to certain embodiments described herein;

FIG. 11 illustrates principal component analysis or factor analysis according to certain embodiments described herein;

FIG. 12 illustrates a wrapper-based feature ordering chart according to certain embodiments described herein;

FIG. 13 illustrates the averaged best F1-scores according to certain embodiments described herein;

FIG. 14 illustrates a chart showing feature selection according to certain embodiments described herein;

FIG. 15 illustrates a graph showing Bayesian information criterion during wrapper feature selection according to certain embodiments described herein;

FIG. 16 illustrates a graph showing performance metrics according to certain embodiments described herein;

FIG. 17 illustrates a graph showing performance metrics according to certain embodiments described herein;

FIG. 18 illustrates a graph showing a RNN and LSTM architectures according to certain embodiments described herein;

FIG. 19 illustrates cross-validation performance according to certain embodiments described herein; and

FIG. 20 illustrates a decision threshold table according to certain embodiments described herein.

### DETAILED DESCRIPTION

There remains a need for systems and methods that can help with early diagnosis of CKD in dogs or canines. Certain non-limiting embodiments, therefore, can process one or more biomarkers or demographic information of the dog using a prediction model. The processed one or more biomarkers or demographic information can be used to determine a probability risk score of the dog for developing CKD. Based on the probability risk score, a customized recommendation can be determined to help reduce the health risks associated with CKD. For clarity and not by way of limitation, the detailed description of the presently disclosed subject matter is divided into the following subsections:
1. Definitions;
2. Biomarkers;
3. Prediction model;
4. Customized recommendations; and
5. Device and system.

### 1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the methods and systems of the disclosure and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The terms “comprises,” “comprising,” or any other variation thereof, are intended to cover a non-exclusive inclusion, such that a process, method, article, system, or apparatus that comprises a list of elements does not include only those elements but can include other elements not expressly listed or inherent to such process, method, article, or apparatus.

The terms “embodiment,” “an embodiment,” “one embodiment,” “in various embodiments,” “certain embodiments,” “some embodiments,” “other embodiments,” “certain other embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with any other embodiment whether or not explicitly described.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The term “effective treatment” or “effective amount” of a substance means the treatment or the amount of a substance that is sufficient to effect beneficial or desired results, including clinical results, and, as such, an “effective treatment” or an “effective amount” depends upon the context in which it is being applied. In the context of administering a composition to reduce a risk of CKD, and/or administering a composition to treat or delay the progression of CKD, an effective amount of a composition described herein is an amount sufficient to treat and/or ameliorate CKD, as well as decrease the symptoms and/or reduce the likelihood of developing CKD. An effective treatment described herein is a treatment sufficient to treat and/or ameliorate CKD, as well as decrease the symptoms and/or reduce the likelihood of CKD. The decrease can be a 10%, 20%, 30%, 40%, 50%, 60%, 70%, 80%, 90%, 95%, 98% or 99% decrease in severity of symptoms of CKD, or likelihood of CKD. An effective amount can be administered in one or more administrations. A likelihood of an effective treatment described herein is a probability of a treatment being effective, i.e., sufficient to treat and/or ameliorate CKD, as well as decrease the symptoms.

As used herein, and as well understood in the art, “treatment” is an approach for obtaining beneficial or desired results, including clinical results. For purposes of this subject matter, beneficial or desired clinical results include, but are not limited to, alleviation or amelioration of one or more symptoms, diminishment of extent of disease, stabilized (i.e., not worsening) state of disease, prevention of disease, reducing the likelihood of developing disease, delay or slowing of disease progression, and/or amelioration or palliation of the disease state. The decrease can be a 10%, 20%, 30%, 40%, 50%, 60%, 70%, 80%, 90%, 95%, 98% or 99% decrease in severity of complications or symptoms. “Treatment” can also mean prolonging survival as compared to expected survival if not receiving treatment.

The terms “animal” or “pet” as used in accordance with the present disclosure refers to domestic animals including, but not limited to, domestic dogs, domestic cats, horses, cows, ferrets, rabbits, pigs, rats, mice, gerbils, hamsters, goats, and the like. Domestic dogs and cats are particular non-limiting examples of pets. The term “animal” or “pet” as used in accordance with the present disclosure can further refer to wild animals, including, but not limited to bison, elk, deer, venison, duck, fowl, fish, and the like.

The terms “pet product,” “pet food,” “pet food composition,” “pet food product,” and/or “final pet food product” means any product or composition that is intended for use or consumption by an animal, such as a cat, a dog, a guinea pig, a rabbit, a bird or a horse. For example, but not by way of limitation, the animal can be a “domestic” dog or canine. A “pet product” “pet food” or “pet food composition” or “pet food product” or “final pet food product” includes any food, feed, snack, food supplement, liquid, beverage, treat, toy (chewable and/or consumable toys), meal, meal substitute or meal replacement. In certain embodiments, the “pet product” can provide certain health or nutritional benefits to the animal.

As used herein, the term “decision threshold” can refer to a predefined or predetermined value or level used in the diagnosis of CKD. The “decision threshold,” for example, can range from about 0.0 to about 1. In certain embodiments, a “decision threshold” of 0.5 can be used. In certain embodiments, the “decision threshold” value can be derived by evaluating or balancing one of more of F1-score, precision, accuracy, sensitivity, and/or specificity. The “decision threshold,” for example, can be a sliding scale where the trade-off or balancing between one or more of F1-score, precision, accuracy, sensitivity, and/or specificity can be determined based on clinical needs or applications.

The term “biomarker” means a characteristic that is objectively measured and evaluated as an indicator of normal biological processes, pathogenic processes, or pharmacologic responses to a therapeutic intervention. The term “biomarker” can also mean any substance, structure, or process that can be measured in the body or its products and influence or predict the incidence of outcome or disease. For example, the biomarker can be analyzed or determined from a urine or blood sample of a dog. Examples of the biomarker can include, but are not limited to, alkaline phosphatase, amylase, protein, BUN or urea level, creatinine, phosphorus, calcium, urine protein, potassium, glucose, hematocrit, hemoglobin, red blood cell (RBC) count, red cell distribution width (RDW), alanine aminotransferase, albumin, bilirubin, chloride, cholesterol, eosinophil, globulin, lymphocyte, monocyte, mean corpuscular hemoglobin (MCH), mean corpuscular hemoglobin concentration (MCHC), mean corpuscular volume (MCV), mean platelet volume (MPV), platelet count, segmented neutrophils, sodium, urine PH level, and/or white blood cell count. In certain non-limiting embodiments, the one or more biomarkers can be obtained from the blood, urine, serum, plasma, or saliva of the dog or canine.

The term “training dataset” means a database of unique dogs or canines that can be used to train the prediction model. In some non-limiting embodiments, the “training dataset” can include demographic information, such as age, weight, breed, and reproductive status of the dog. The age can include the age of a pet during a visit to the veterinarian and/or the age of the pet during the first diagnosis of CKD. In certain non-limiting embodiments, the “training dataset” can include one or more biomarkers.

The term “visit” means a meeting between a healthcare practitioner or provider, such as a veterinarian, and a dog. In certain embodiments, a medical record is generated during or after a visit. In certain embodiments, an amount of one or more biomarkers is determined during a visit. In certain embodiments, a diagnosis of CKD is made during a visit. The practitioner can make a visit to a hospital and/or in a home or other location. A dog or canine, taken by an owner, can make a visit to the practitioner in a clinic or an office.

The term “urine specific gravity” (a.k.a. urine SG or USG) measures the ratio of urine density compared to water density. It is a measure of the concentration of solutes in the urine, and it provides information on the ability of a kidney to concentrate urine.

The term “customized recommendation” means any treatment, method, or test used to lower/reduce the risk of developing CKD or reducing/managing the symptoms or effects of CKD. For example, the “customized recommendation” can include one or more therapeutic interventions, one or more dietary recommendations, one or more renal sparing strategies, or one or more tests for disease progression.

### 2. Biomarkers

In certain non-limiting embodiments, one or more biomarkers or demographic information of a dog or canine can be used, in part, to determine a probability risk score for developing CKD.

In certain embodiments disclosed herein, the one or more biomarker can be used for predicting CKD based on one or more biological parameters related to the development of CKD. The customized recommendation can then be tailored depending on the risk of developing CKD indicated by the biomarkers.

In certain embodiments, BUN and urea measurement is interchangeable. As BUN reflects only the nitrogen content of urea (molecular weight 28) and urea measurement reflects the whole molecule (molecular weight 60), urea measurement is 2.14 (60/28) times of BUN measurement.

In certain non-limiting embodiments, the biomarker can include one or more of the following: urine specific gravity level in a urine sample of a dog or canine; total creatinine level in the blood of the dog or canine; creatinine level in the serum of the dog or canine; creatinine in the plasma of the dog or canine; the creatinine in a urine sample of the dog or canine; the urine protein in a urine sample of the dog or canine; the total urea in the blood of the dog or canine; the urea in the serum of the dog or canine; the urea in the plasma of the dog or canine; the urea in a urine sample of the dog or canine; the BUN or urea in the blood of the dog or canine; the white blood cell count (WBC) in the blood of the dog or canine; the urine pH in a urine sample of the dog or canine. In certain non-limiting embodiments, a change in a level of a biomarker can be associated with an increased risk of developing CKD.

With each biomarker, an increased or a decreased level of the biomarker can give information about a dog or canine’s susceptibility to developing CKD, depending on the particular biomarker. For example, in certain embodiments, a decreased level of urine specific gravity indicates an increased risk of developing CKD. In certain embodiments, an increased level of urine specific gravity indicates a decreased risk of developing CKD. In certain embodiments, a lower level of urine specific gravity compared to a predetermined reference value based on average levels of urine specific gravity in the population of dogs or canines in the dataset can indicate an increased risk of developing CKD. In certain embodiments, a higher level of urine specific gravity compared to a predetermined reference value based on average levels of urine specific gravity in a control population indicates a decreased risk of developing CKD. In certain embodiments, the average levels of urine specific gravity in the dataset population is between about 1.00 and about 1.1, between about 1.01 and about 1.09, between about 1.02 and about 1.08, or between about 1.03 and about 1.07. In certain embodiments, the average levels of urine specific gravity in a control population is between about 1.001 and about 1.08. In certain embodiments, the predetermined reference value of urine specific gravity is about 100%, about 99%, about 98%, about 97%, about 96%, about 95%, about 94%, about 93%, about 92%, about 91%, about 90%, about 89%, about 88%, about 87%, about 86%, about 85%, about 80%, about 75%, about 70% or less, or any intermediate percentage or range of the average level of urine specific gravity in a control population in the dataset. In certain embodiments, the predetermined reference value of urine specific gravity is between about 99.9% and about 90%, between about 95% and about 90%, or between about 99% and about 92% of the average level of urine specific gravity in a control population in the dataset. In certain embodiments, the predetermined reference value of urine specific gravity is between about 1.001 and about 1.08, between about 1.001 and about 1.07, between about 1.001 and about 1.06, between about 1.001 and about 1.05. or between about 1.001 and about 1.04. In certain embodiments, a dog or canine’s hydration status can be considered to adjust the urine specific gravity level.

In certain non-limiting embodiments, an increased level of creatinine can indicate an increased risk of developing CKD. A decreased or lowered level of creatinine can indicate a decreased risk of developing CKD. A higher level of creatinine can indicate an increased risk of developing CKD. For example, the average levels of creatinine in a dataset can be set between about 0 mg/dL and about 3 mg/dL, between about 0.8 mg/dL and about 3 mg/dL, between about 1 mg/dL and about 2.8 mg/dL, or between about 1.2 mg/dL and about 2.2 mg/dL. In certain embodiments, the average levels of creatinine in a control population is between about 0.8 mg/dL and about 2.4 mg/dL, In certain embodiments, the predetermined reference value of creatinine can be about 100%, about 105%, about 110%, about 115%, about 120%, about 125%, about 130%, about 140%, about 150%, about 200%, about 250%, about 300%, about 400%, about 500% or more, or any intermediate percentage or range of the average level of creatinine in a control population. In certain embodiments, the predetermined reference value of creatinine can be between about 100% and about 120%, between about 120% to about 150%, between about 150% and about 200%, or between about 200% and about 500% of the average level of creatinine in a control population. In certain non-limiting embodiments, the predetermined reference value of creatinine can be between about 0 mg/dL and about 3 mg/dL, between about 1 mg/dL and about 2.4 mg/dL, between about 1 mg/dL and about 2 mg/dL, or between about 1.2 mg/dL and about 1.8 mg/dL.

In certain embodiments, a decreased level of urine protein can indicate an increased risk of developing CKD. An increased level of urine protein can indicate a decreased or increased risk of developing CKD. A decreased level of urine protein can indicate an increased or decreased risk of developing CKD. In certain embodiments, a lower level of urine protein compared to a predetermined reference value, based on average levels of urine protein, can indicate an increased risk of developing CKD. In certain embodiments, a higher level of urine protein, compared to a predetermined reference value, based on average levels of urine protein in a control population in the dataset indicates a decreased risk of developing CKD. A higher level of urine protein can indicate infection or kidney damage. In certain non-limiting embodiments, a historic bout of elevated urine protein can indicate earlier infections and/or higher risk of kidney damage. In certain non-limiting embodiments, current elevation of urine protein indicates higher risk of declining renal function and/or CKD. A dog or canine can exhibit a higher level of urine protein compared to a predetermined reference value. For example, a higher level of urine protein is found in a current sample of the dog or canine or in a recent medical record of the dog or canine (e.g., a record made within about 1 week, about 2 weeks, about 3 weeks, about 4 weeks, about 5 weeks, about 10 weeks, about 3 months or about 6 months before practicing any one of the methods disclosed herein). In certain embodiments, a dog or canine has exhibited a higher level of urine protein compared to a predetermined reference value in the past. For example, a higher level of urine protein is found in a historic sample of the dog or canine or in a historical medical record of the dog or canine (e.g., a record made more than about 1 week, about 2 weeks, about 1 month, about 2 months, about 3 months or about 6 months before practicing any one of the methods disclosed herein). In certain embodiments, the average levels of urine protein in a control population can be between about 0 mg/dL and about 50 mg/dL, between about 0 mg/dL and about 25 mg/dL, between about 0 mg/dL and about 10 mg/dL, or between about 0 mg/dL and about 5 mg/dL. In certain embodiments, the average levels of urine protein in a control population is between about 48 to 50 mg/dL. In certain embodiments, the predetermined reference value of urine protein can be at least about 100%, about 110%, about 120%, about 130%, about 140%, about 150%, about 160%, about 170%, about 180%, about 190%, about 200%, about 250%, about 300%, about 400%, about 500%, about 1000%, about 2000%, about 5000%, about 10000% or more, or any intermediate percentage or range of the average level of urine protein in a control population. In certain embodiments, the predetermined reference value of urine protein can be between about 100% and about 200%, between about 200% and about 500%, or between about 200% and about 1000% of the average level of urine protein in a control population. In certain embodiments, the predetermined reference value of urine protein is between about 0.001 mg/dL and about 100 mg/dL, between about 1 mg/dL and about 80 mg/dL, between about 5 mg/dL and about 70 mg/dL, between about 10 mg/dL and about 60 mg/dL, or between about 20 mg/dL and about 50 mg/dL.

In certain embodiments, an increased level of BUN or urea indicates an increased risk of developing CKD. In certain embodiments, a decreased level of BUN or urea indicates a decreased risk of developing CKD. In certain embodiments, a higher level of BUN or urea compared to a predetermined reference value based on average levels of BUN or urea in a control population can indicate an increased risk of developing CKD. In certain embodiments, a lower level of BUN or urea compared to a predetermined reference value based on average levels of BUN or urea in a control population can indicate a decreased risk of developing CKD. In certain embodiments, the average levels of BUN in a control population is between about 5 mg/dL and about 100 mg/dL, between about 10 mg/dL and about 55 mg/dL, between about 15 mg/dL and about 40 mg/dL, or between about 20 mg/dL and about 30 mg/dL. In certain embodiments, the average levels of BUN in a control population is between about 17 mg/dL and about 55 or 56 mg/dL. In certain embodiments, the predetermined reference value of BUN or urea can be about 100%, about 105%, about 110%, about 115%, about 120%, about 125%, about 130%, about 140%, about 150%, about 200%, about 250%, about 300%, about 400%, about 500% or more, or any intermediate percentage or range of the average level of BUN or urea in a control population. In certain embodiments, the predetermined reference value of BUN or urea can be between about 100% and about 120%, between about 120% to about 150%, between about 150% and about 200%, or between about 200% and about 500% of the average level of BUN or urea in a control population. In certain embodiments, the predetermined reference value of BUN is between about 10 mg/dL and about 100 mg/dL, between about 15 mg/dL and about 90 mg/dL, between about 17 mg/dL and about 56 mg/dL, between about 20 mg/dL and about 80 mg/dL, between about 30 mg/dL and about 70 mg/dL, or between about 40 mg/dL and about 60 mg/dL.

In certain non-limiting embodiments, a decreased level of WBC can indicate an increased risk of developing CKD. In certain non-limiting embodiments, an increased level of WBC indicates an increased or decreased risk of developing CKD. In certain non-limiting embodiments, a decreased level of WBC indicates a decreased or increased risk of developing CKD. In certain non-limiting embodiments, WBC can be used by a prediction model to rule out other infections, or by one or more prediction models to relate previous infections to future risk. For example, WBC can be used by a prediction model to understand dehydration level and normalize the values of other biomarkers. In some non-limiting embodiments, a prediction model can be generated by machine learning algorithm, such as a recurrent neural network or LTSM, as described below. The prediction model can interpret the WBC count according to any current and/or previous values of other biomarkers. In certain non-limiting embodiments, a higher level of WBC compared to a predetermined reference value based on average levels of WBC in a control population can indicate an increased risk of developing CKD. In certain other non-limiting embodiments, a higher level of WBC can indicate infection or kidney damage. A historic bout of elevated WBC, for example, can indicate earlier infections and/or higher risk of kidney damage. In another example, the current elevation of WBC can indicate higher risk of declining renal function and/or CKD. In certain non-limiting embodiment, a dog or canine can exhibit a higher level of WBC compared to a predetermined reference value. The higher level of WBC can be found in a current sample or medical record of the dog or canine (e.g., a record made within about 1 week, about 2 weeks, about 3 weeks, about 4 weeks, about 5 weeks, about 10 weeks, about 3 months or about 6 months before practicing any one of the methods disclosed herein). In some non-limiting embodiments, a dog or canine has exhibited a higher level of WBC compared to a predetermined reference value in the past. The higher level of WBC can be found in a historic sample or medical record of the dog or canine (e.g., a record made more than about 1 week, about 2 weeks, about 1 month, about 2 months, about 3 months or about 6 months before practicing any one of the methods disclosed herein). In certain non-limiting embodiments, the average levels of WBC in a control population is between about 1 x 10⁹ /L and about 60 x 10⁹ /L, between about 2 x 10⁹ /L and about 50 x 10⁹ /L, between about 5 x 10⁹ /L and about 30 x 10⁹ /L, between about 6 x 10⁹ /L and about 20 x 10⁹ /L or between about 8 x 10⁹ /L and about 16 x 10⁹ /L. In certain embodiments, the average levels of WBC in a control population can be between about 13.5 x 10⁹ /L. In certain embodiments, the predetermined reference value of WBC can be about 100%, about 105%, about 110%, about 115%, about 120%, about 125%, about 130%, about 140%, about 150%, about 200%, about 250%, about 300%, about 400%, about 500% or more, or any intermediate percentage or range of the average level of WBC in a control population. In certain embodiments, the predetermined reference value of WBC can be between about 100% and about 120%, between about 120% to about 150%, between about 150% and about 200%, or between about 200% and about 500% of the average level of WBC in a control population. For example, in certain non-limiting embodiments the predetermined reference value of WBC can be between about 2 x 10⁹ /L and about 100 x 10⁹ /L, between about 5 x 10⁹ /L and about 80 x 10⁹ /L, between about 10 x 10⁹ /L and about 70 x 10⁹ /L, between about 20 x 10⁹ /L and about 60 x 10⁹ /L or between about 30 x 10⁹ /L and about 50 x 10⁹ /L. In certain non-limiting embodiments, a lower level of WBC can indicate a decreased risk of developing CKD. In certain embodiments, the predetermined reference value of WBC can be about 100%, about 95%, about 90%, about 85%, about 80%, about 75%, about 70%, about 60%, about 50% or less, or any intermediate percentage or range of the average level of WBC in a control population. In certain embodiments, the predetermined reference value of WBC can be between about 100% and about 90%, between about 80% and about 60%, or between about 60% and about 40% of the average level of WBC in a control population.

In certain embodiments, a decreased level of urine pH indicates an increased risk of developing CKD. In certain embodiments, an increased level of urine pH can indicate a decreased risk of developing CKD, while a lower level of urine pH can indicate an increased risk of developing CKD. In certain embodiments, a higher level of urine pH can indicate a decreased risk of developing CKD. In some non-limiting embodiments, the average levels of urine pH in a control population of the dataset can be between about 4 and about 8.5, between about 5 and about 8, between about 5.2 and about 7.5, or between about 6 and about 7. In particular, the average levels of urine pH in a control population can be between about 5.5 and about 7.5. In certain non-limiting embodiments, the predetermined reference value of urine pH can be about 100%, about 95%, about 90%, about 85%, about 80%, about 75%, about 70%, about 60%, about 50% or less, or any intermediate percentage or range of the average level of urine pH in a control population. In certain embodiments, the predetermined reference value of urine pH can be between about 100% and about 80%, between about 80% and about 60%, or between about 60% and about 40% of the average level of urine pH in a control population. In certain embodiments, the predetermined reference value of urine pH can be between about 3 and about 8, between about 4 and about 7.5, between about 4.5 and about 7, between about 4.5 and about 6.5, between about 5 and about 6.5, or between about 5 and about 6. In certain embodiments, a dog or canine’s diet and the handling of the urine sample of the dog or canine can, in part, contribute to the adjustment of the urine specific gravity level.

In certain non-limiting embodiments, an increased or a decreased level of a biomarker can be detected in a current sample or in a recent medical record of a dog or canine (e.g., a record made within about 1 week, about 2 weeks, about 3 weeks, about 4 weeks, about 5 weeks, about 10 weeks, about 3 months or about 6 months before practicing any one of the methods disclosed herein). In some non-limiting embodiments in which the dog or canine has exhibited an increased or a decreased level of a biomarker in the past. For example, an increased or a decreased level of urine protein can be found in a historic sample of the dog or canine or in a historical medical record of the dog or canine (e.g., a record made more than about 1 week, about 2 weeks, about 1 month, about 2 months, about 3 months, about 6 months, about 12 months, about 2 years, about 3 years, about 4 years, about 5 years, and/or any time between, prior, or after, before practicing any one of the methods disclosed herein).

In general, the ranges of average levels for the biomarkers can account for 50 to 100% of the healthy, normal population. For some biomarkers, the ranges of average levels for the biomarkers can account for 80 to 95%. Therefore, about 5-25% of the population can have values above the higher end of an average/normal range, and about another 5-25 % of the population can have values below the low end of an average/normal range. In certain embodiments, the actual ranges and validity of the biomarkers can be determined by each laboratory or testing, depending on the machine and/or on the population of dogs or canines tested to determine an average/normal range. Additionally, laboratory tests can be impacted by sample handling and machine maintenance/calibration. Updates to machines can also result in changes in the normal ranges. Any one of these factors can be considered for adjusting the average levels and/or the predetermined reference values of each biomarker.

Beyond the above described biomarkers, certain non-limiting embodiments can include one or more of the following biomarkers: phosphate and parathyroid hormone (PTH), symmetric dimethylarginine (SDMA), systolic blood pressure, potassium, total calcium, hyaluronic acid, death receptor 5, transforming growth factor β1, ferritin, beta globin, catalase, alpha globin, epidermal growth factor receptor pathway substrate 8, mucin isoform precursor, ezrin, delta globin, moesin, phosphoprotein isoform, annexin A2, myoglobin, hemopexin, serine proteinase inhibitor, serpine peptidase inhibitor, CD14 antigen precursor, fibronectin isoform preprotein, angiotensinogen preprotein, complement component precursor, carbonic anhydrase, uromodulin precursor, complement factor H, complement component 4 BP, heparan sulfate proteoglycan 2, olfactomedian-4, leucine rich alpha-2 glycoprotein, ring finger protein 167, inter-alpha globulin inhibitor H4, heparan sulfate proteoglycan 2, N-acylshingosine aminohydrolase, serine proteinase inhibitor clade A member 1, mucin 1, clusterin isoform 1, brain abundant membrane attached signal protein 1, dipeptidase 1, fibronectin 1 isoform 5 preprotein, angiotensinogen preproprotien, carbonic anhydrase, uromodulin precursor, Metalloproteinase inhibitor 2, Insulin-like growth factor-binding protein 7, Immunoglobulin A, Immunoglobulin G1, Immunoglobulin G2, Alpha-1 antitrypsin, Serum amyloid P component, Hepatocyte growth factor, Intercellular adhesion molecule 1, Beta-2-glycoprotein 1, Interleukin-1 beta, Neutrophil Elastase, Tumor necrosis factor receptor superfamily member 11B, Interleukin-11, Cathepsin D, C—C motif chemokine 24, C—X—C motif chemokine 6, C—C motif chemokine 13, C—X—C motif chemokines -1, -2, and -3, Matrilysin, Interleukin-2 receptor alpha chain, Insulin-like growth factor-binding protein 3, Macrophage colony-stimulating factor 1, apolipoprotein C—I, apolipoprotein C—II, fibrinogen alpha chain, fibrinogen A-alpha chain, kininogen, Inter-Alpha Inhibitor H4 (ITIH4), keratin Type I cytoskeletol 10 cystatin A, cystatin B, and any combination thereof. See, for example, U.S. Publication No. 2012/0077690 A1, U.S. Publication No. 2013/0323751 A1, EP 3,112,871 A1, EP 2,462,445 A1, and EP 3,054,301 A1.

In certain non-limiting embodiments, the amounts of the biomarkers in the dog or canine can be detected and quantified by any means known in the art. In certain other non-limiting embodiments, the level of creatinine, urine protein, WBC, urea and/or BUN can be determined by a fluorescence method or a luminescence method. In certain embodiments, the level of creatinine, urine protein, WBC, urea and/or BUN can be determined by an antibody-based detection method, such as an enzyme-linked immunosorbent assay (ELISA) or a sandwich ELISA.

In other examples, the level of urine protein can be determined by using a urine albumin antibody, the level of urine specific gravity can be measured by refractometry, hydrometry, and/or reagent strips. On the other hand, in certain non-limiting embodiments, the level of urine pH can be measured by a pH test strip, or a pH meter and a pH probe, while the level of WBC can be measured by flow cytometry.

In certain non-limiting embodiments, other detection methods, such as other spectroscopic methods, chromatographic methods, labeling techniques, and/or quantitative chemical methods can be used. The level of a biomarker from a dog or canine and/or a predetermined reference value of the biomarker can be determined by the same method.

### 3. Prediction Model

Some non-limiting embodiments can be directed to method or systems for identifying susceptibility of a dog to develop CKD. The method can include processing, or the system be caused to process, at least one of the one or more biomarkers or demographic information of the dog using a prediction model. The prediction model can be trained using one or more machine learning techniques.

### 3.1 Dataset

For example, the prediction model can be trained using the following dataset:

TABLE 1

Summary of Dataset at Time of Evaluation (T0)

| Dataset | Training | | Test | |
|---|---|---|---|---|
| Diagnosis Group | CKD | No CKD | CKD | No CKD |
| Number of dogs 15128 | | 22082 | 7430 | 11275 |
| Mean visits per dog | 15.47 | 12.02 | 15.57 | 11.97 |
| Male to female ratio | 1:1.1 | 1:0.93 | 1:1.09 | 1:1.09 |
| Mean (SD) age (years) at T0 | 11.56 (3.48) | 7.21 (2.98) | 11.55 (3.49) | 7.15 (2.96) |
| Mean (SD) weight (kg) at T0 | 12.94 (11.12) | 15.12 (12.41) | 1328 (11.29) | 14.74 (12.03) |
| Mean (SD) nitrogen (mg/dL) at T0 | 56.31 (32.77) | 17.38 (5.54) | 55.94 (32.81) | 17.47 (5.65) |
| Mean (SD) creatinine (mg/dL) at T0 | 2.66 (1.85) | 1.09 (0.29) | 2.66 (1.9) | 1.08 (0.27) |
| Mean (SD)urine protein (mg/dL) at T0 | 90.16 (179.43) | 49.50 (133.88) | 92.71(186.38) | 48.34 (142.11) |
| Mean (SD) Urine SG at T0 | 1.020 (0.011) | 1.039 (0.012) | 1.020 (0.011) | 1.039 (0.012) |
| Percent missing creatinine | 11.5% | 7.4% | 11.0% | 7.9% |
| Percent missing Urine SG | 57.0% | 60.0% | 57.0% | 60.1% |

The dataset shown in Table 1 can include one or more biomarkers and/or demographic information of a dog. For example, the demographic information can include the age or weight of the pet at the time of visit, age of the pet at the time CKD was first diagnosed, and/or the gender of the dog or canine. The one or more biomarkers shown in Table 1 can include BUN, creatinine, urine protein, urine SG. One or more other biomarkers, such as amylase, or other demographic information can be included in the dataset.

In certain non-limiting embodiments, a dataset, which can be referred to as a training dataset, can include medical records of a plurality of dogs or canines. For example, the training dataset described in Table 1 ranges from about 15,128 to about 22,082 dogs or canines. In another example, the test dataset described in Table 1 ranges from about 7,430 to about 11,275 dogs or canines. In other embodiments the number of dogs or canines included in a dataset can be between 100 to 100,000 different dogs or canines, such as 55,885 dogs or canines. The medical records, for example, can include an amount of one or more biomarkers and/or demographic information of the dog or canine. In certain embodiments, the medical records can include one or more visits of a dog or canine. For example, the training and test datasets described in Table 1 range from about 11 to about 16 visits per dog or canine. In other non-limiting embodiments, however, the number of visits can range from 1 to 100 visits per dog or canine. In certain non-limiting embodiments, the medical records can include the most recent two, three, four, or five visits of a dog or canine at different time points. In another non-limiting embodiments, the medical records can include records of the first and the last visits of a dog or canine at different time points.

In some non-limiting embodiments, the training dataset can be stratified, formed, or arranged for cross validation purposes. Cross validation can be used to help assess how the results of the prediction model can generalize to an independent dataset. A dataset, for example, can be divided or stratified into 2 or more folds where one or more subsets can be used to validate the prediction model by one or more different subsets. In certain embodiments, the training dataset is stratified into about 3, 4, 5, 6, 7, 8, 9, 10, 20, 30, 40, or 50 folds.

In certain non-limiting embodiments, rather than being stratified for cross validation purposes the dataset can be divided into subsets for one or more different prediction models. The subset, for example, can correspond to those dogs or canines diagnosed with CKD during a given visit, or diagnosed with CKD 3 months, 6 months 12, months, 2 years, 3 years, 4 years, or 5 years after a given visit. In other non-limiting embodiments, the training dataset can be divided into any other subsets.

In some non-limiting embodiments, if a medical record or chart of a dog or canine is missing a value, amount, or level of one or more biomarkers and/or demographic information, the missing amount, level, or demographic information can be imputed. Missing data can be based on one of the following: data missing completely at random (MCAR) when the probability of an instance having a missing value for a variable does not depend on either the known values or the missing data; data missing at random (MAR) when the probability of an instance having a missing value for a variable can depend on the known values but not on the value of the missing data itself; data missing not at random (MNAR) when the probability of an instance having a missing value for a variable can depend on the value of that variable.

The missing values can be imputed, which can mean that the missing value is replaced with a plausible value. The imputation, in certain non-limiting embodiments, can be calculated using statistical methods or processes, such as mean, median regression multiple, or ridge regression imputation. In a mean or median imputation approach the missing components of a vector can be filled in by the average value or median value of that component. In some non-limiting embodiments, a matrix factorization method or process can be used for imputing the missing values. For example, the matrix factorization method or process can include UV matrix factorization, soft-impute, iterative singular value decomposition (SVD) imputation, or biscaler plus soft-impute.

In other non-limiting embodiments, imputation can be calculated using machine learning. For example, the imputed value, amount, level, or demographic information can be determined using one of more of the following machine learning methods: k-nearest neighbor (KNN) imputation, such as missingpy KNN or fancyimpute KNN, multiple or multivariant imputation by chained equations (MICE) imputation, such as linear regression, ridge regression, or gradient boost, and/or random forest algorithms or related algorithms, such as missingpy missForest, sciblox MICE random forest, or any other variant of missing forest. The metrics used for measuring the imputation can include, for example, root mean squared error (RMSE), mean absolute error (MAE) metrics, and normalized RMSE.

RMSE, for example, can be calculated using the following equation:

$$\sqrt{\frac{1}{N} {\sum }_{i}^{N} {\left({\text{y}}_{i} - {x}_{i}\right)}^{2}} ,$$

where N is the number of the missing values, yᵢ is the imputed value, and xᵢ is the true value. MAE, for example, can be calculated using the following equation:

$$\frac{1}{N} {\sum }_{i}^{N} \left|{y}_{i} - {x}_{i}\right| ,$$

where N is the number of the missing values, yᵢ is the imputed value, and xᵢ is the true value. Normalized RMSE, for example, can be calculated using the following equation:

$$\sqrt{\frac{m e a n {\left({X}^{t r u e} - {X}^{i m p}\right)}^{2}}{v a r \left({X}^{t r u e}\right)}} ,$$

where X^{true} can be the complete data matrix, and X^{imp} can be the imputed data matrix. The var() can be the variance computed over the continuous missing values.

In certain non-limiting embodiments, for imputing all 34 features, including the one or more biomarkers and/or demographic information, the missForest and MICE imputation, in particular the linear regression, were the top performing imputations based on RMSE and MAE, with missForest being better in 75% of the experiments. On the other hand, for imputing the six features, including the one or more biomarkers and/or demographic information, such as urine protein, urine specific gravity, urine PH, BUN, creatinine, or WBC, MICE imputation performed better than missForest. For example, for imputing the six features the top performing imputation for RMSE can be fancyipute MICE, sciblox MICE libear, and/or sciblox MICE boost. In another example, for imputing the six features the top performing imputation for MAE can be fancyipute MICE, sciblox MICE libear, and/or sciblox MICE boost.

In some non-limiting embodiments, visiting age can be beneficial for imputing the six features when either the block of urine analyte values (e.g., urine protein, urine specific gravity, urine PH) or the block of blood values (e.g., BUN, creatinine, WBC) are missing. Specifically, the visiting age of the dog or canine can help to increase imputation accuracy. The improvement in the block of blood values can be larger when accounting for visiting age than the block of urine analyte values. For example, the median MEA gain can be 1.1% for blood and 0.2% for urine.

The chosen imputation method or process can be based on the amount of missing information in the dataset. For example, for datasets with 10% missing values MICE linear regression can be used, whereas for datasets with 20% or 30% missing values missForest can be used.

In certain non-limiting embodiments, the training dataset can be filtered by a set of inclusion and exclusion criteria. For example, a visit count of a dog or canine, such as no less than 2, no less than 3, no less than 4, or no less than 5 visits, can be used as an inclusion or exclusion criteria. In another example, the medical history of visits or the visit age of the dog or canine can be used as inclusion or exclusion criteria.

In certain non-limiting embodiments, the dataset may include a total of 55,885 dogs or canines. The dataset may also exclude information collected from dogs or canines before the age of 1.5 and after the age of 22 years. The dataset may also include a diverse population of dogs or canines, including mixed breed dogs and/or over 280 pedigree breeds. One or more biomarkers and/or demographic information were selected from the dataset as features for a CKD prediction model. In one example, 35 biomarkers and/or pieces of demographic information were selected. The dataset may also include dogs or canines diagnosed with CKD and dogs or canines who have not been diagnosed with CKD.

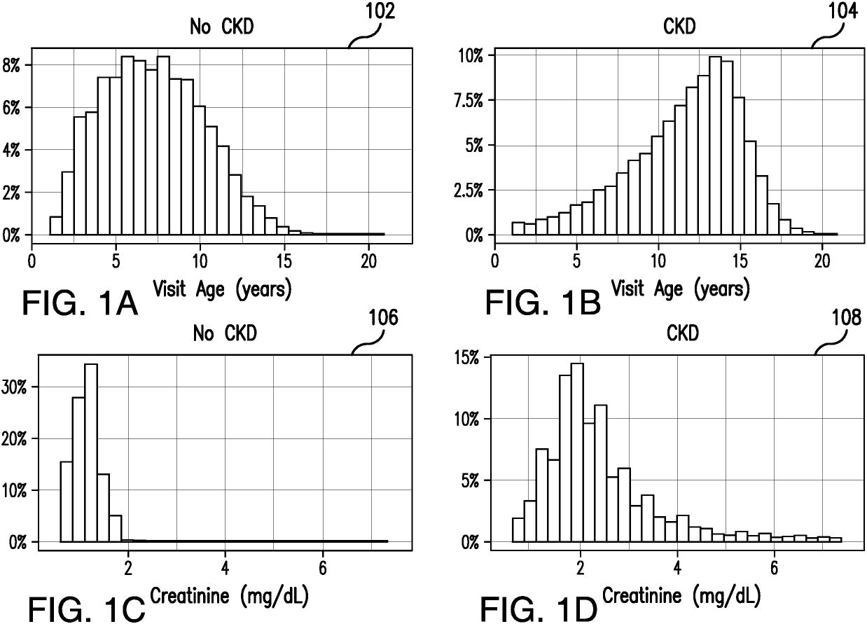

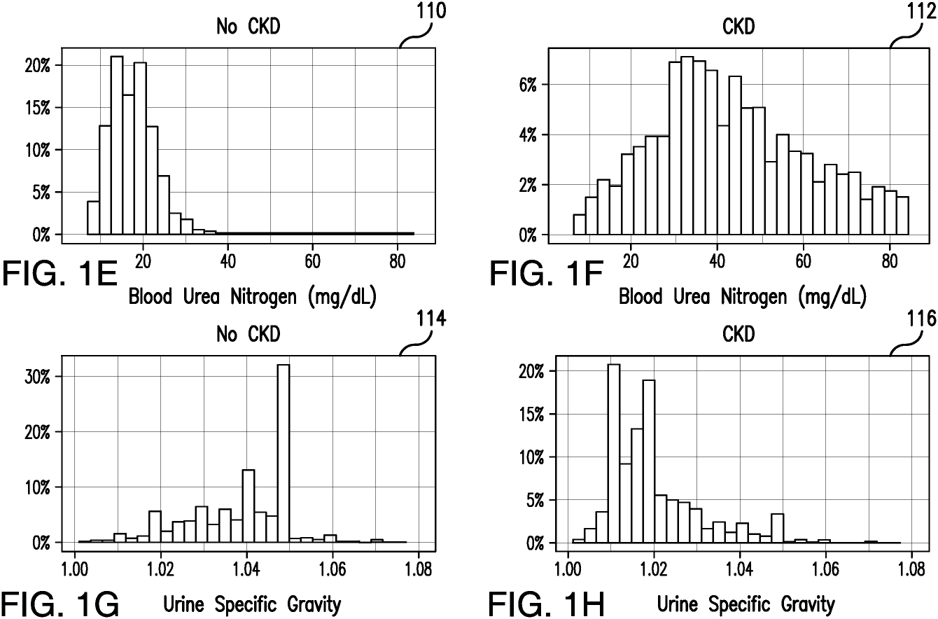

FIGS. 1A - 1H illustrate distribution charts of the study dataset according to certain embodiments described herein. In particular, FIGS. 1A - 1H illustrate biomarkers and demographic information for both dogs diagnosed with CKD, shown in FIGS. 1B, 1D, 1F, and 1H, and dogs not diagnosed with CKD, as shown in FIGS. 1A, 1C, 1E, and 1G. The age T(0) in FIGS. 1A - 1H can be the age at which the dog or canine was first or originally diagnosed with CKD. In some non-limiting embodiments, data collected more than 30 days after the first or original diagnosis was excluded, and/or an additional 30-day window was included to capture serum, blood, or urine test data that was entered into the database shortly after the diagnosis visit.

The dataset shown in FIGS. 1A - 1H can exclude dogs or canines without a formal CKD diagnosis, but that have at least two-CKD suggesting data points, such as blood creatinine above normal values and/or urine-specific gravity below normal values. Other CKD suggesting data-points can include one or more of the following terms appearing in the medical notes of the health records: “CKD,” “azotemic,” “Royal Canin Veterinary diet Renal,” or “Hill’s prescription diet k/d.” The CKD suggesting data-points can be based on blood or urine test results.

All other pets or canines not diagnosed with CKD or having at least two-CKD suggesting data points, and having at least two year of data, were included in the dataset and assigned a “no CKD” status, as shown in FIGS. 1A, 1C, 1E, and 1G. For those “no CKD” dogs or canines, the T(0) was set as the age of the last visit minus two years.

In some non-limiting embodiments, the health records can be further filtered based on information content by imposing that the dogs or canines should have had at least 2 visits with blood creatinine data. This resulted in a final study data set of 55,915 dogs or canines, of which 22,558 dogs were diagnosed with CKD and the remaining 33,357 had “no CKD.” The “no CKD” dogs or canines can be referred to as the control group. As shown in the graphs of FIGS. 1A - 1H, graphs 102, 106, 110, and 114 can represent visit age, creatinine level, BUN, and urine specific gravity, respectively, of those dogs or canines assigned a “no CKD” label. On the other hand, graphs 104, 108, 112, and 116 represent visit age, creatinine level, BUN, and urine specific gravity, respectively, of those dogs or canines diagnosed with CKD. Those dogs or canines diagnosed with CKD were older, had higher BUN levels, and/or lower urine specific gravity, as illustrated in FIGS. 1B, 1F, and 1H. The results support the quality of the CKD diagnosis within the dataset and provide confidence in the data used to build the model.

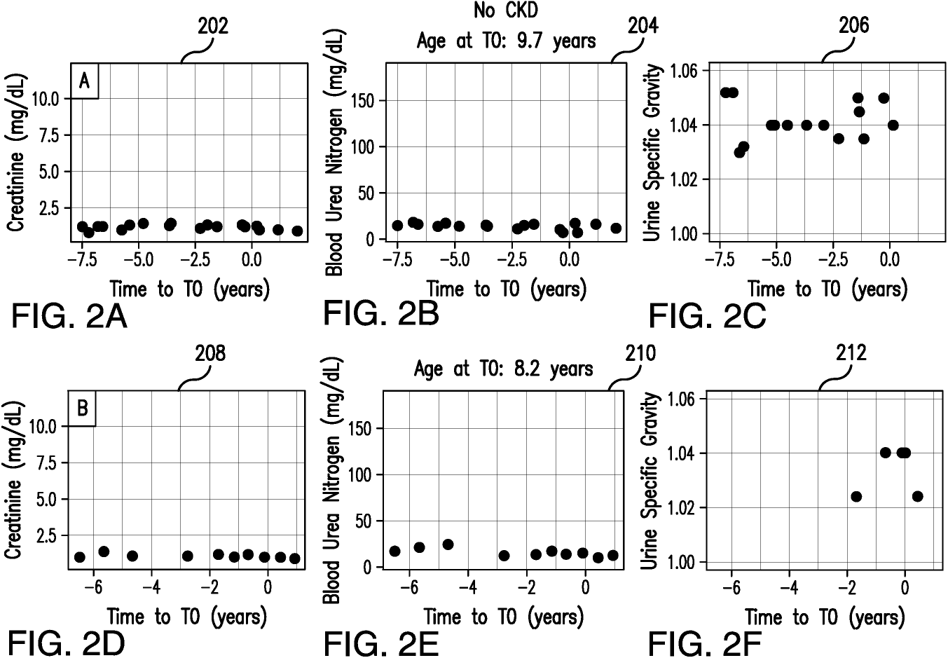

FIGS. 2A - 2L illustrate example EHRs with “no CKD” according to certain embodiments described herein. In particular, FIGS. 2A - 2L illustrate observations of individual dogs or canines for creatinine, blood urea nitrogen and urine specific gravity as a function of time before T(0). Graphs 202, 208, 214, and 220 shown in FIGS. 2A, 2D, 2G, and 2J, can illustrate creatinine as a function of time before T(0). Graphs 204, 210, 216, and 222 shown in FIGS. 2B, 2E, 2H, and 2K, can illustrate BUN as a function of time before T(0). Graphs 206, 212, 218, and 224 shown in FIGS. 2C, 2F, 2I, and 2L, can illustrate urine specific gravity as a function of time before T(0). Age T(0) can be 9.7 years at graphs 202, 204, and 206 shown in FIGS. 2A, 2B, and 2C, age T(0) can be 6.2 years at graphs 208, 210, and 212 shown in FIGS. 2D, 2E, 2F, age T(0) can be 9.5 years at graphs 214, 216, and 218 shown in FIGS. 2G, 2H, and 2I, and age T(0) can be 9.2 years at graphs 220, 222, and 224 shown in FIGS. 2J, 2K, and 2L.

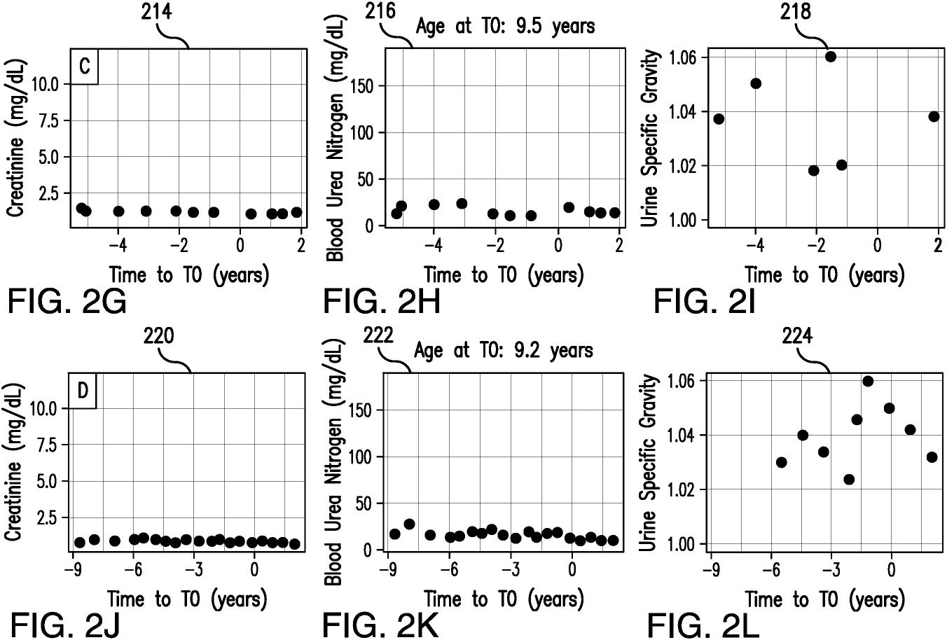

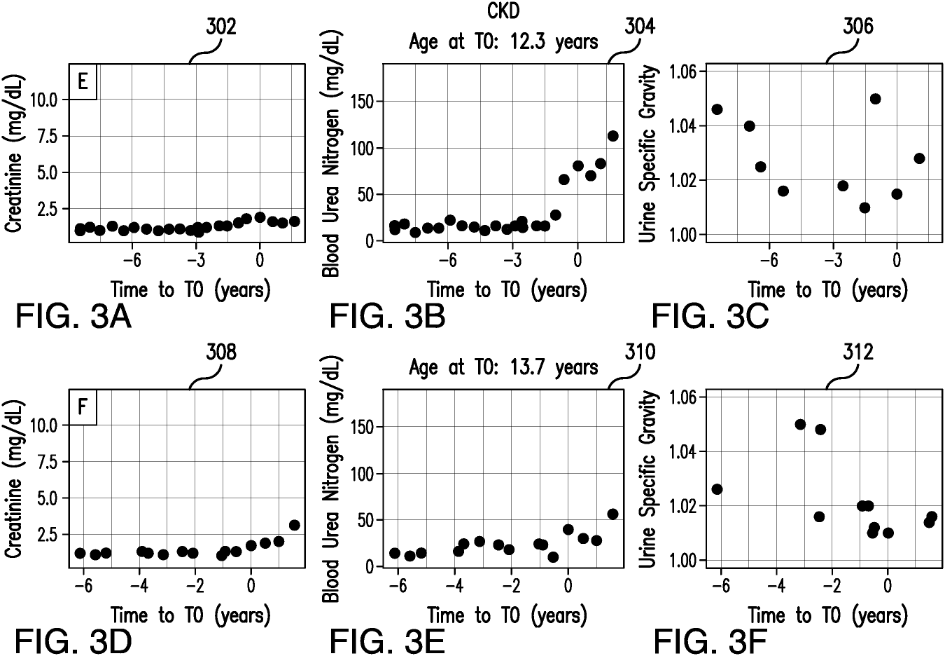

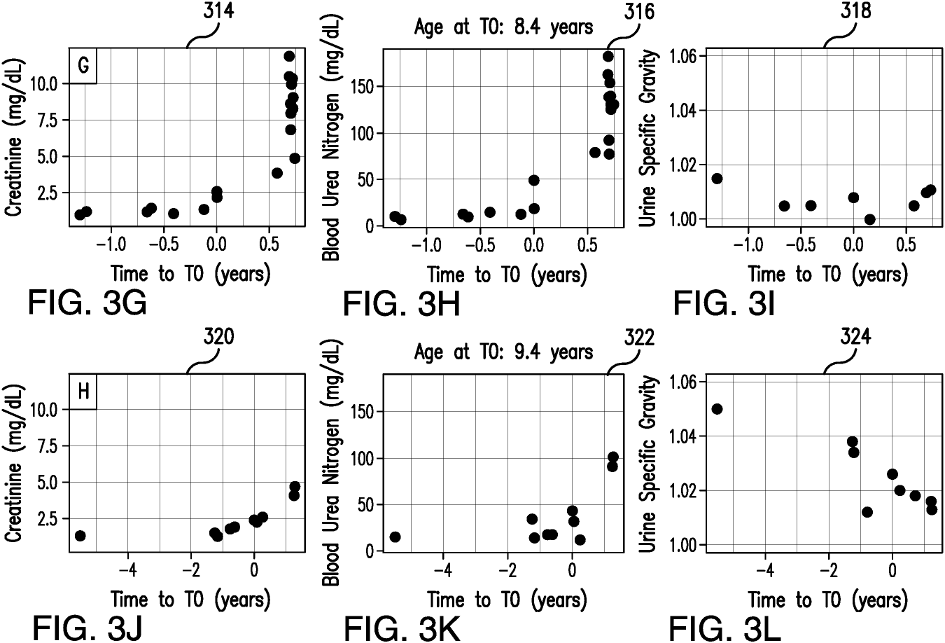

FIGS. 3A - 3L illustrate example EHRs with CKD according to certain embodiments described herein. In particular, FIGS. 3A - 3L illustrate observations of individual dogs or canines for creatinine, blood urea nitrogen and urine specific gravity as a function of time before T(0). Graphs 302, 308, 314, and 320 shown in FIGS. 3A, 3D, 3G, and 3J, can illustrate creatinine as a function of time before T(0). Graphs 304, 310, 316, and 322 shown in FIGS. 3B, 3E, 3H, and 3K, can illustrate BUN as a function of time before T(0). Graphs 306, 312, 318, and 324 shown in FIGS. 3C, 3F, 3I, and 3L, can illustrate urine specific gravity as a function of time before T(0). Age T(0) can be 12.3 years at graphs 302, 304, and 306 shown in FIGS. 3A - 3C, age T(0) can be 13.7 years at graphs 308, 310, and 312 shown in FIGS. 3D - 3F, age T(0) can be 8.4 years at graphs 314, 316, and 318 shown in FIGS. 3G - 3I, and age T(0) can be 9.4 years at graphs 320, 322, and 324 shown in FIGS. 3J - 3L.

In the samples shown in FIGS. 2A - 2L and 3A - 3L, the “no CKD” differs from the CKD. There can be considerable heterogeneity within the latter groups with many changes happening before the time of diagnosis. This helps to illustrate that a prediction model should not only consider multiple factors at the time of diagnosis, but also include information at different time points before diagnosis.

To determine the CKD prediction model, the dataset was randomly split into two parts. For example, as shown in Table 1, of 55,915 total health records, 37,210 health records, or approximately 67% of the data, were used to build the CKD prediction model. The remaining 18,705 health records, or approximately 33% of the data, were used as a test dataset to evaluate model performance or for cross validation. The model building dataset, also known as a training dataset, and the model testing dataset can be kept separate throughout the analysis to exclude any bias at the testing stage. Prior to use, missing information in the blood and urine testing dataset can be imputed using all available blood and/or urine data, but not the CKD status information. In certain non-limiting embodiments, the missing information can be imputed because the neural network can require complete data. In some datasets, the prevalence of missing data can be about 10% for most of the blood chemistry measures, and/or about 60% for urine test results. The model building dataset and testing dataset were kept separate to avoid any flow of information between the datasets.

### 3.2 Type of Prediction Models

In certain non-limiting embodiments, the prediction model for dog or canine CKD can include one or more machine learning algorithms. The intrinsically multifactorial nature of canine CKD presents an ideal setting for prediction models to add clinical value. For example, the machine learning algorithm can be supervised, such as logistic regression or back propagation neural networks. In other examples, the machine learning algorithm can be unsupervised, such as an Apriori algorithm or K-means clustering, semi-supervised, or reinforcement, such as using a Q-learning algorithm, or temporal difference learning. In some other non-limiting embodiments, any other suitable learning style can be used.

In such embodiments in which prediction model utilizes a machine learning algorithm, the machine learning algorithm can include, for example, one of the following algorithms or methods: a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naive Bayes, averaged one-dependence estimators, Bayesian belief network, etc.), a kernel method (e.g., a support vector machine, a radial basis function, a linear discriminate analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a deep learning algorithm (e.g., a restricted Boltzmann machine, a deep belief network method, a convolution network method, a stacked auto-encoder method, etc.), a dimensionality reduction method (e.g., principal component analysis, partial lest squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), a condition random field algorithm and any suitable form of algorithm.

In certain non-limiting embodiments, prediction model can include one or more of a logistic regression algorithm, an artificial neural network algorithm (ANN), a recurrent neural network algorithm (RNN), a K-nearest neighbor algorithm (KNN), a KNN with dynamic time warping (KNN-DTW), a Naive Bayes algorithm, a support vector machine algorithm (SVM), a random forest algorithm, an AdaBoost algorithm, and/or any combination thereof. In some non-limiting embodiments, a regularization algorithm can be used. The regularized algorithm, for example, can help to prevent overfitting.

In certain non-limiting embodiments, the prediction model can be an RNN including algorithm comprising an input layer, an output layer, and/or one or more hidden layers. The RNN, for example, can be a vanilla RNN, a long short-term memory (LSTM) RNN, and/or a gated recurrent unit (GRU) RNN. In some non-limiting embodiments, the RNN can include 1 to 50 hidden layers, such as 1, 2, 3, 4, 5, 6, 7, 8, 9, or 10 hidden layers. Each input layer, output layer, or hidden layer can include 1 to 500 nodes. Some non-limiting embodiments, for example, can include 1, 2, 3, 4, 5, 6, 7, 8, 9, or 10 nodes. Each of the input, output, or hidden layer can include either a same or different number of nodes.

In certain embodiments, the one or more hidden layers can include an activation function. The activation function helps to determine the output of a given node in the one or more hidden layers. The activation function, for example, can be a TanH function, a sigmoid or logistic function, a rectified linear units (ReLU) function, a maxout function, or a guassian function. Any other activation function known in the art can be used as part of the prediction model.

As described above, in certain non-limiting embodiments the prediction model can be trained using KNN with dynamic time warping (DTW). The one or more biomarkers and/or the demographic information processed by the prediction model can be selected by a filter method, such as a Pearson correlation coefficient. In some non-limiting embodiments, the one or more biomarkers and/or demographic information can be selected by a top-down wrapper method KNN-DTW, using a K or nearest neighbor between about 7 to 17. In certain other non-limiting embodiments, the one or more biomarkers and/or the demographic information can be selected by a bottom-up wrapper. In other non-limiting embodiments, a mixture of experts (MOE) approach can be employed to train the prediction model, where an ensemble of predictors can be combined using simple or weighted voting.

In certain non-limiting embodiments, the classification algorithm can be trained using an RNN, such as a vanilla RNN, LSTM RNN, or GRU RNN, comprising an input layer, an output layer and one or more hidden layer, with each hidden layer having one or more nodes. For example, the RNN can include three hidden layers, the first layer including five nodes, the second layer including three nodes, and the third layer including three nodes. The RNN can utilize a cross validation process that includes about 1 to about 100-fold cross validation process. Further, the RNN can be trained over about 1 to 100 epochs. For example, an RNN can include a ten-fold cross-validation process and can be trained over 8 or 18 epochs.

The input layer of the prediction model can include one or more biomarkers and/or demographic information of the dog or canine. The output layer of the prediction model can include a softmax or normalized exponential function. The softmax function can help to normalize the output of the prediction model into a probability distribution, with the number of probabilities being proportional to the exponentials of the input values. In some non-limiting embodiments, a binary cross-entropy can also be used for loss calculation. Other non-limiting embodiments can utilize a regularization algorithm to prevent overfitting. The regularization algorithm, for example, can cause about 5%, 10%, 15%, 20%, 25%, 30%, 35%, 40%, or any other percentage to dropout to avoid overfitting.

In certain non-limiting embodiments, the prediction model can include assessment or validation of the prediction model. The assessment or validation, in some non-limiting embodiments, can be used to update the prediction model. For example, the prediction model can include a ten-fold cross validation. As part of the cross validation, the dataset can be stratified into about 2 folds, about 3 folds, about 4 folds, about 5 folds, about 6 folds, about 7 folds, about 8 folds, about 9 folds, about 10 folds, about 20, about 30 folds, about 40 folds, about 50 folds or more folds, or any intermediate number of folds for cross validation.

In some non-limiting embodiments, performance of the prediction model can be characterized by an area under the curve (AUC) ranging from about 0.50 to about 0.99.

The prediction model can be used to determine a probability risk score for the dog for developing CKD. The probability risk score, for example, can be based on a probability of a dog or canine developing a CKD. The probability risk score, for example, can be any value between 0 and 100% or between 0.0 and 1.0. Based on the determined probability risk score, a dog or canine can be determined to have a high risk of developing CKD with low or high certainty. The low or high certainty can be based on at least one of the accuracy, sensitivity, specificity, or F1 score of the probability risk score. For example, an accuracy of 95% or more can be said to be at a high risk of developing CKD, while an accuracy between 25 to 50% can be said to be at a low risk of developing CKD.

In certain non-limiting embodiments, the dog or canine can have a low certainty or a high certainty of not developing CKD. For example, an accuracy of about 80% or less can indicate a low certainty for the dog or canine to be at no risk of developing CKD. An accuracy of about 80% or more can indicate a high certainty for the dog or canine to be at no risk of developing CKD. In some non-limiting embodiments, the dog or canine with a low certainty to be at no risk of developing CKD can be classified as a future CKD. In some non-limiting embodiments, a high probability risk score can indicate that the dog or canine will develop CKD with a high predictable accuracy. For, the high predictable accuracy can be more than about 95%.

In some non-limiting embodiments, a medium probability risk score can indicate inconclusive or insufficient data to accurately predict the susceptibility of a dog or canine to develop or not develop CKD. A medium probability score, for example, can be a score between 40% and 60%, or any other value that does not indicate either a high or low probability. On the other hand, a low probability risk score can indicate that the dog or canine cannot develop CKD with a high predictable accuracy. For example, the high predictable accuracy can be more than about 95%.

The probability risk score can relate to the risk of the dog or canine developing CKD within about 0 month, about 3 months, about 6 months, about 9 months, about 12 months, 0 year, about 0.5 year, about 1 year, about 2 years, about 3 years, about 4 years, or about 5 or more years after the amount of value of the one or more biomarkers is determined, or after the determination of the probability score. In certain other examples, the probability score can indicate the risk of the dog or canine will develop CKD within about 12 months or about 2 years after the amount of value of the one or more biomarkers is determined, or after the determination of the probability score.

In certain non-limiting embodiments, a prediction model, such as an RNN, can be used to process one or more biomarkers, such as creatinine, BUN, urine specific gravity, urine protein, or demographic information, as well as demographic information, such as weight or age. In one example, the prediction model near the point of diagnosis can display a sensitivity of about 91.4% and a specificity of 97.2%. The specificity of the prediction model can remain at about 97% one year before or two year before diagnosis, while the sensitivity can fall to about 69% and 45% respectively. The prediction model can help provide early diagnosis of CKD, allowing greater opportunities for interventions and patient outcomes.

To determine the CKD prediction model, the dataset was randomly split into two parts. In total 37,210 health records, or approximately 67% of the data, were used to build the CKD prediction model. The remaining 18,705 health records, or approximately 33% of the data, were used as a test dataset to evaluate model performance or for cross validation. The model building dataset, also known as a training dataset, and the model testing dataset can be kept separate throughout the analysis to exclude any bias at the testing stage. Prior to use, missing information in the blood and urine testing dataset can be imputed using all available blood and/or urine data, but not the CKD status information. In certain non-limiting embodiments, the missing information can be imputed because the neural network can require complete data. The model building dataset and testing dataset were kept separate to avoid any flow of information between the datasets.

In certain non-limiting embodiments, for the prediction model to work well for early detection of CKD, the model predicting building dataset can be augmented by adding truncated versions of the original health records. For example, the last K visits can be removed with K ranging from 1 to the total number of visits minus 1. Truncating the data, in some embodiments, can help to enrich the dataset with health records having a gap of up to 2 years between the last visit in the dataset and the time of diagnosis. The truncated data can then help to train the prediction model with more pet subsets having a large gap in their data between the last available data point and the date of diagnosis.

In some non-limiting embodiments, one of the initial steps in building a CKD prediction model can be to select a limited set of model features. The features can include one or more biomarkers and/or demographic information. Feature selection, for example, can be conducted by a top-down or bottom-up wrapper method using a standard recurrent neural network. The initially tested recurrent neural network can have two hidden layers, the first layer having 3 nodes and the second layer having 7 nodes. The RNN, in some embodiments, can include a TanH activation function in the hidden layers, as well as a softmax for transforming the output layer into a CKD probability risk score. In certain non-limiting embodiments, backpropagation through time can be used for training the RMSprop gradient optimization algorithm. Model performance, for example, can be evaluated based on F1 cross-entropy in a 3-fold cross-validation setup. The F1 cross-entropy can be used as a metric for balancing sensitivity and specificity, independent of the CKD incidence.

A full model architecture screen can then be performed using the selected features. Different RNN configurations of 1 to 5 hidden layers were then tested with 3 to 200 nodes per layer. In some non-limiting embodiments, a 20% dropout was added to avoid overfitting. The evaluation, for example, was based on an F1 score in a 10-fold cross-validation setup. The prediction model configuration can be fine-tuned with respect to the training time in the same cross-validation set-up.

In certain non-limiting embodiments, unbiased model performance can be assessed by applying the selected prediction model to the testing dataset. Predictions were performed for all dogs or canines in the CKD and “no CKD” groups. The results of the prediction model were interpreted at the crude model output, such as the probability of a CKD diagnosis, and/or at the after categorization output, in which “no CKD” and CKD are assigned using a p of 0.5 as a cut-out point. Categorical results, for “CKD” and “no CKD” groups, were used to compute sensitivity estimates (i.e., proportion of true positives, “CKD” status predicted as CKD) and specificity estimates (i.e., proportion of true negatives, “no CKD” predicted as no CKD), respectively. Confidence intervals for the sensitivity and specificity estimates were calculated using normal approximation. The ability for the model to predict CKD ahead of the definitive diagnosis can be evaluated by truncating the health records to various time points before age at evaluation (T0) for the CKD group, and/or allowing the prediction model to only see the truncated data.

As discussed above, in some non-limiting embodiments a standard RNN with a 3-7 hidden layer structure can be used as a starting point for a prediction model for CKD. The stating prediction model can acknowledge both the multifactorial and temporal aspects of CKD diagnosis. Using this prediction model with 35 candidate factors, including one or more biomarkers and/or demographic information. In certain non-limiting embodiments, the most important features, such as the one or more biomarkers and/or demographic information, can be selected using a top-down and bottom-up feature selection strategy. In some non-limiting embodiments, the cross-entropy score improved by adding up to 6 features and plateaued thereafter. The best feature set, for example, can be creatinine, blood urea nitrogen, urine specific gravity, urine protein, weight and age. Using these 6 features, an updated prediction model can be determined or selected. In certain non-limiting embodiments, a three-layer RNN with a 5-3-3 structure can perform best, trained over 8 epochs.

### 4. Customized Recommendation

In certain non-limiting embodiments, a customized recommendation can be determined based on the probability risk of the dog or canine developing CKD. The customized recommendation can be transmitted to a user equipment of a veterinarian, owner, or caregiver of the dog or canine. In general, the customized recommendation can provide a method, process, test, or regimen for treating, preventing, or reducing a risk of developing CKD for a dog or canine. For example, the customized recommendation can include at least one of one or more therapeutic interventions, one or more dietary changes, one or more renal sparing strategies, and/or one or more tests for disease progression.

In certain non-limiting embodiments, the customized recommend can include testing for disease progression, such as testing of serum parathyroid hormone levels. When the probability risk score is a low probability score or indicates no risk of developing CKD with high certainty, the customized recommendation can include testing for CKD within one or more weeks, months, or years of the probability risk score calculation. For example, the customized recommendation can include testing for CKD a year or two after the original probability risk score is determined. In some non-limiting embodiments, when a medium probability score is determined, or indicates no risk of developing CKD with low certainty, the customized recommendation can include testing the dog or canine for CKD within 6 months after the original probability risk score is determined. In other non-limiting embodiments, when the probability risk score has a medium or high probability score, or indicates risk of developing CKD with low certainty, the customized recommendation can include testing the dog or canine for CKD within 3 months after the original probability risk score is determined.

When the probability risk score indicates with high certainty a risk of developing CKD, the customized recommendation can include identifying underlying commodities, testing the dog or canine for CKD, and/or continuing with International Renal Interest Society (IRIS) staging disclosed herein.

For example, when the probability risk score of the dog or canine indicates a risk of developing CKD with high certainty, the customized recommendation can include a therapeutic intervention. The therapeutic intervention can include at least one of monitoring water consumption and litter box habits, providing a dietary regimen, providing a supplemental recommendation, providing high quality diet with no protein restriction and appropriate phosphorus levels, considering providing fatty acid supplement, avoiding nephrotoxic drugs, implementing dental care regimen, and/or maintaining good oral health.

In certain non-limiting embodiments, the customized recommendation can include diagnosing the presence of a comorbidity in the dog or canine. In certain embodiments, the comorbidity can include one or more of the following: hyperthyroidism, diabetes mellitus, hepatopathy, underweight, murmur, arthritis, malaise, constipation, gastroenteritis, vomiting, inflammatory bowel disease, crystalluria, enteritis, urinary tract infection, upper respiratory disease, urinary tract disease, obesity, inappropriate elimination, cystitis, colitis, and/or any combination thereof. In particular, in some non-limited embodiments the comorbidity can include hyperthyroidism, diabetes mellitus, hepatopathy, underweight, murmur, and/or any combination thereof.

In certain embodiments, the customized recommendation can include a therapeutic intervention or a renal sparing strategy. For example, the therapeutic intervention or renal sparing strategy can include one or more of the following: avoidance of non-steroidal anti-inflammatories or aminoglycosides, hemodialysis, renal replacement therapy, withdrawal of kidney damaging compounds, kidney transplantation, delaying or avoiding kidney damaging procedures, modifying diuretic administration, and/or any combination thereof. In certain other non-limiting embodiments, the therapeutic intervention or renal sparing strategy can include one or more of the following: reducing phosphate intake, reducing protein intake, administering polyunsaturated fatty acids, administering a phosphate binder therapy, administering potassium, reducing dietary sodium intake, administering alkali supplements, or any combinations thereof. See for example, Jonathan D. Foster, Update on Mineral and Bone Disorders in Chronic Kidney Disease, Vet Clin North Am Small Anim Pract. 2016 Nov;46(6): 1131-49.

In certain embodiments, the customized recommendation can include a dietary recommendation, such as a nutritional recommendation, a dietary or nutritional change, a dietary or nutritional regimen, a nutritional product, and/or a dietary or nutritional therapy. The dietary recommendation can include the recommendation to use any pet product, and/or the intake or use of any pet product, such as a pet food. For example, can include one or more of the following: a low phosphorus diet, a low protein diet, a low sodium diet, a potassium supplement diet; a polyunsaturated fatty acid (PUFA, e.g., long chain omega-3 fatty acids) supplement diet, an anti-oxidant supplement diet, a vitamin B supplement diet, a liquid diet, a calcium supplement diet, a regular protein diet, or any combinations thereof. In certain other embodiments, the dietary recommendation can include one or more pet products. The pet products, for example, can help to delay the onset, limit the progress, reduce the effects, minimize the physiological burden, or prevent CKD. For example, the diet can include a low protein, low phosphorus, increase calcium to phosphorus ratio, ,an increased energy density, and/or a neutral acid-base balance.

In certain embodiments, a low phosphorus diet can include between about 0.01% and about 5%, between about 0.1% and about 2%, between about 0.1% and about 1%, between about 0.05% and about 2%, or between about 0.5% and about 1.5% phosphorus on a weight by weight basis of a pet food. In certain non-limiting embodiments, a low phosphorus diet can include about 0.01%, about 0.05%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5% phosphate, or any intermediate percentage or range of phosphate on a weight by weight basis of a pet food. In some non-limiting embodiments, a low phosphorus diet can include about 0.1 g/1000 kcal, about 0.2 g/1000 kcal, about 0.3 g/1000 kcal, about 0.4 g/1000 kcal, about 0.5 g/1000 kcal, about 0.6 g/1000 kcal, about 0.7 g/1000 kcal, about 0.8 g/1000 kcal, about 0.9 g/1000 kcal, about 1.0 g/1000 kcal, about 1.1 g/1000 kcal, about 1.2 g/1000 kcal, about 1.3 g/1000 kcal, about 1.4 g/1000 kcal, about 1.5 g/1000 kcal, about 1.6 g/1000 kcal, about 1.7 g/1000 kcal, about 1.8 g/1000 kcal, about 1.9 g/1000 kcal, about 2.0 g/1000 kcal, about 2.1 g/1000 kcal, about 2.2 g/1000 kcal, about 2.5 g/1000 kcal, about 2.8 g/1000 kcal, about 3.0 g/1000 kcal, about 3.5 g/1000 kcal, about 4 g/1000 kcal, about 5 g/1000 kcal, about 10 g/1000 kcal, about 15 g/1000 kcal, about 20 g/1000 kcal, or any intermediate percentage or range of phosphate. In certain other non-limiting embodiments, a low phosphorus diet can include between about 0.1 g/1000 kcal and about 0.5 g/1000 kcal, between about 0.5 g/1000 kcal and about 1.0 g/1000 kcal, between about 1.0 g/1000 kcal and about 2.0 g/1000 kcal, between about 2.0 g/1000 kcal and about 5.0 g/1000 kcal, between about 0.01 g/1000 kcal and about 0.1 g/1000 kcal, between about 0.05 g/1000 kcal and about 1.0 g/1000 kcal, between about 0.1 g/1000 kcal and about 1 g/1000 kcal, between about 0.1 g/1000 kcal and about 2 g/1000 kcal, between about 1 g/1000 kcal and 2 g/1000 kcal of phosphate. In certain non-limiting embodiments, a low phosphorus diet can include about 0.5% phosphate on a weight by weight basis of a pet food. (e.g., about 1.2 g/1000 kcal for the dry renal diet or about 1.0 g/1000 kcal for the wet renal diet). In other examples, a low phosphorus diet can include about 0.9 or 1% phosphate on a weight by weight basis of a pet food (e.g., about 1.8 g/1000 kcal for the dry maintenance diet or about 2.3 g/1000 kcal for the wet maintenance diet). A low phosphorus diet can also include between about 1.0 g/1000 kcal and about 1.5 g/1000 kcal of phosphorus.

In certain non-limiting embodiments, a calcium supplement diet can include between about 0.01% and about 5%, between about 0.1% and about 2%, between about 0.1% and about 1%, between about 0.05% and about 2%, or between about 0.5% and about 1.5% calcium on a weight by weight basis of a pet food. In some non-limiting embodiments, a calcium supplement diet can include about 0.01%, about 0.05%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5% calcium, or any intermediate percentage or range of calcium on a weight by weight basis of a pet food. In some other non-limiting embodiments, a calcium supplement diet can include about 0.1 g/1000 kcal, about 0.2 g/1000 kcal, about 0.3 g/1000 kcal, about 0.4 g/1000 kcal, about 0.5 g/1000 kcal, about 0.6 g/1000 kcal, about 0.7 g/1000 kcal, about 0.8 g/1000 kcal, about 0.9 g/1000 kcal, about 1.0 g/1000 kcal, about 1.1 g/1000 kcal, about 1.2 g/1000 kcal, about 1.3 g/1000 kcal, about 1.4 g/1000 kcal, about 1.5 g/1000 kcal, about 1.6 g/1000 kcal, about 1.7 g/1000 kcal, about 1.8 g/1000 kcal, about 1.9 g/1000 kcal, about 2.0 g/1000 kcal, about 2.1 g/1000 kcal, about 2.2 g/1000 kcal, about 2.5 g/1000 kcal, about 2.8 g/1000 kcal, about 3.0 g/1000 kcal, about 3.5 g/1000 kcal, about 4 g/1000 kcal, about 5 g/1000 kcal, about 10 g/1000 kcal, about 15 g/1000 kcal, about 20 g/1000 kcal, or any intermediate percentage or range of calcium. In certain other non-limiting embodiments, a calcium supplement diet can include between about 0.1 g/1000 kcal and about 0.5 g/1000 kcal, between about 0.5 g/1000 kcal and about 1.0 g/1000 kcal, between about 1.0 g/1000 kcal and about 2.5 g/1000 kcal, between about 2.5 g/1000 kcal and about 5.0 g/1000 kcal, between about 0.01 g/1000 kcal and about 0.1 g/1000 kcal, between about 0.05 g/1000 kcal and about 1.0 g/1000 kcal, between about 0.1 g/1000 kcal and about 1 g/1000 kcal, between about 0.1 g/1000 kcal and about 2 g/1000 kcal, between about 1 g/1000 kcal and 2 g/1000 kcal of calcium.

In certain non-limiting embodiments, a combinatory calcium supplement and low phosphorus diet can include a calcium-phosphorus ratio (Ca:P ratio) of between about 1 and about 2, between about 1.1 and about 1.4, between about 1.2 and about 1.4, between about 1.1 and about 1.3, between about 1.3 and about 1.8, between about 1.4 and about 1.6, between about 1.5 and about 1.8, or between about 1.6 and about 1.8. In some non-limiting embodiments, a combinatory calcium supplement and low phosphorus diet can include a calcium-phosphorus ratio (Ca:P ratio) of about 1, about 1.1, about 1.2, about 1.3, about 1.4, about 1.5, about 1.6, about 1.7, about 1.8, about 1.9, or about 2.0.

In certain non-limiting embodiments, a low sodium diet can include between about 0.00001% and about 5%, between about 0.0001% and about 1%, between about 0.001% and about 0.1%, or between about 0.001% and about 0.05% sodium on a weight by weight basis of a pet food. In some non-limiting embodiments, a low sodium diet can include about 0.01%, about 0.05%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5% sodium, or any intermediate percentage or range of sodium on a weight by weight basis of a pet food. A low sodium diet can also include about 1 mg/kg/day, about 2 mg/kg/day, about 3 mg/kg/day, about 4 mg/kg/day, about 5 mg/kg/day, about 6 mg/kg/day, about 7 mg/kg/day, about 8 mg/kg/day, about 9 mg/kg/day, about 10 mg/kg/day, about 15 mg/kg/day, about 20 mg/kg/day, about 30 mg/kg/day, about 40 mg/kg/day, about 46 mg/kg/day, about 50 mg/kg/day, about 60 mg/kg/day, about 70 mg/kg/day, about 80 mg/kg/day, about 90 mg/kg/day, about 100 mg/kg/day about 120 mg/kg/day, about 150 mg/kg/day, or any intermediate amount or range of sodium. In other non-limiting embodiments, a low sodium diet comprises between about 1 mg/1000 kcal and about 50 mg/1000 kcal, between about 2 mg/1000 kcal and about 20 mg/1000 kcal, between about 5 mg/1000 kcal and about 50 mg/1000 kcal, between about 1 mg/1000 kcal and about 10 mg/1000 kcal, between about 0.1 mg/1000 kcal and about 5 mg/1000 kcal, between about 0.1 mg/1000 kcal and about 10 mg/1000 kcal, between about 0.1 mg/1000 kcal and about 20 mg/1000 kcal, between about 0.1 mg/1000 kcal and about 40 mg/1000 kcal, between about 10 mg/1000 kcal and 20 mg/1000 kcal of sodium. A low sodium diet, for example, can include about 0.4 to about 0.9 mmol/kg/day, or about 9.2 to about 20.7 mg/kg/day.

In certain non-limiting embodiments, a potassium supplement diet can include between about 0.00001% and about 5%, between about 0.0001% and about 1%, between about 0.001% and about 0.1%, or between about 0.001% and about 0.05% potassium supplement on a weight by weight basis of a pet food in addition to the potassium existing in the pet food. In other non-limiting embodiments, a potassium supplement diet can include about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5% or more potassium supplement on a weight by weight basis of a pet food in addition to the potassium existing in the pet food, or any intermediate percentage or range of potassium supplement in addition to the potassium existing in a pet food on a weight by weight basis of a pet food. In some non-limiting embodiments, a potassium supplement diet can include about 1 mg/kg/day, about 2 mg/kg/day, about 3 mg/kg/day, about 4 mg/kg/day, about 5 mg/kg/day, about 6 mg/kg/day, about 7 mg/kg/day, about 8 mg/kg/day, about 9 mg/kg/day, about 10 mg/kg/day, about 15 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day or more, or any intermediate amount or range of potassium supplement in addition to the potassium existing in a pet food. In certain embodiments, a potassium supplement diet can include between about 1 mg/1000 kcal and about 10 mg/1000 kcal, between about 2 mg/1000 kcal and about 20 mg/1000 kcal, between about 5 mg/1000 kcal and about 50 mg/1000 kcal, between about 1 mg/1000 kcal and about 10 mg/1000 kcal, between about 0.1 mg/1000 kcal and about 5 mg/1000 kcal, between about 0.1 mg/1000 kcal and about 10 mg/1000 kcal, between about 0.1 mg/1000 kcal and about 20 mg/1000 kcal, between about 0.1 mg/1000 kcal and about 40 mg/1000 kcal, between about 10 mg/1000 kcal and 20 mg/1000 kcal of potassium supplement in addition to the potassium existing in a pet food.

In certain non-limiting embodiments, a potassium supplement diet can include between about 0.01% and about 5%, between about 0.1% and about 2%, between about 0.1% and about 1%, between about 0.05% and about 2%, or between about 0.5% and about 1.5% potassium on a weight by weight basis of a pet food. In other non-limiting embodiments, a potassium supplement diet can include about 0.01%, about 0.05%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5% potassium, or any intermediate percentage or range of potassium on a weight by weight basis of a pet food. In some non-limiting embodiments, a potassium supplement diet can include about 0.1 g/1000 kcal, about 0.2 g/1000 kcal, about 0.3 g/1000 kcal, about 0.4 g/1000 kcal, about 0.5 g/, about 0.6 g/1000 kcal, about 0.7 g/1000 kcal, about 0.8 g/1000 kcal, about 0.9 g/1000 kcal, about 1.0 g/1000 kcal, about 1.1 g/1000 kcal, about 1.2 g/1000 kcal, about 1.3 g/1000 kcal, about 1.4 g/1000 kcal, about 1.5 g/1000 kcal, about 1.6 g/1000 kcal, about 1.7 g/1000 kcal, about 1.8 g/1000 kcal, about 1.9 g/1000 kcal, about 2.0 g/1000 kcal, about 2.1 g/1000 kcal, about 2.2 g/1000 kcal, about 2.5 g/1000 kcal, about 2.8 g/1000 kcal, about 3.0 g/1000 kcal, about 3.5 g/1000 kcal, about 4 g/1000 kcal, about 5 g/1000 kcal, about 10 g/1000 kcal, about 15 g/1000 kcal, about 20 g/1000 kcal, or any intermediate percentage or range of potassium. A potassium supplement diet can also include between about 0.1 g/1000 kcal and about 0.5 g/1000 kcal, between about 0.5 g/1000 kcal and about 1.0 g/1000 kcal, between about 1.0 g/1000 kcal and about 2.5 g/1000 kcal, between about 2.5 g/1000 kcal and about 5.0 g/1000 kcal, between about 0.01 g/1000 kcal and about 0.1 g/1000 kcal, between about 0.05 g/1000 kcal and about 1.0 g/1000 kcal, between about 0.1 g/1000 kcal and about 1 g/1000 kcal, between about 0.1 g/1000 kcal and about 2 g/1000 kcal, between about 1 g/1000 kcal and 2 g/1000 kcal of potassium. In other non-limiting embodiments, a potassium supplement diet comprises between about 2 g/1000 kcal and about 2.5 g/1000 kcal of potassium. In certain embodiments, a potassium supplement diet comprises about 2.1 g/1000 kcal of potassium.

In certain non-limiting embodiments, a regular protein diet can include a protein level of between about 70 g/1000 kcal and about 90 g/1000 kcal, between about 70 g/1000 kcal and about 75 g/1000 kcal, between about 70 g/1000 kcal and about 80 g/1000 kcal, between about 80 g/1000 kcal and about 90 g/1000 kcal, or between about 85 g/1000 kcal and about 90 g/1000 kcal. In some non-limiting embodiments, a regular protein diet can include a protein level of about 73 g/1000 kcal, about 74 g/1000 kcal, or about 75 g/1000 kcal.

In certain non-limiting embodiments, a low protein diet can include between about 0.0001% and about 20%, between about 0.001% and about 10%, between about 0.01% and about 5%, between about 0.05% and about 2%, or between about 0.01% and about 1% protein on a weight by weight basis of a pet food. In some non-limiting embodiments, a low protein diet can include about 0.01%, about 0.05%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5%, about 10%, about 15%, about 20% protein, or any intermediate percentage or range of protein on a weight by weight basis of a pet food. In other non-limiting embodiments, a low protein diet can include about 1 g/kg/day, about 2 g/kg/day, about 3 g/kg/day, about 4 g/kg/day, about 5 g/kg/day, about 6 g/kg/day, about 7 g/kg/day, about 8 g/kg/day, about 9 g/kg/day, about 10 g/kg/day, about 15 g/kg/day, about 20 g/kg/day or any intermediate amount or range of protein. A low protein diet can also include between about 1 g/kg/day and about 20 g/kg/day, between about 1 g/kg/day and about 50 g/kg/day, between about 2 g/kg/day and about 30 g/kg/day, between about 2 g/kg/day and about 10 g/kg/day, between about 2 g/kg/day and about 8 g/kg/day, between about 5 g/kg/day and about 20 g/kg/day or any intermediate amount or range of protein. A low protein diet can include about 4 to about 6 g/kg/day or about 5 to about 5.5 g/kg/day.

In certain non-limiting embodiments, a polyunsaturated fatty acid (PUFA) supplement diet can include between about 0.01% and about 30%, between about 0.1% and about 20%, between about 1% and about 10%, between about 0.1% and about 5%, or between about 1% and about 10% PUFA supplement in addition to the PUFA existing in a pet food on a weight by weight basis of a pet food. In some non-limiting embodiments, a PUFA supplement diet can include about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5%, about 10%, about 15%, about 20%, about 25%, about 30% or more PUFA supplement in addition to the PUFA existing in a pet food, or any intermediate percentage or range of PUFA supplement in addition to the PUFA existing in a pet food on a weight by weight basis of a pet food. In other non-limiting embodiments, a PUFA supplement diet can include about 0.1 g/kg/day, about 0.5 g/kg/day, about 1 g/kg/day about 1 g/kg/day, about 2 g/kg/day, about 3 g/kg/day, about 4 g/kg/day, about 5 g/kg/day, about 6 g/kg/day, about 7 g/kg/day, about 8 g/kg/day, about 9 g/kg/day, about 10 g/kg/day, about 15 g/kg/day, about 20 g/kg/day, about 30 g/kg/day, about 40 g/kg/day, about 50 g/kg/day, about 60 g/kg/day, about 70 g/kg/day, about 80 g/kg/day, about 90 g/kg/day, about 100 g/kg/day or any intermediate amount or range of PUFA supplement in addition to the PUFA existing in a pet food. In certain other non-limiting embodiments, a PUFA supplement diet can include between about 0.1 g/kg/day and about 20 g/kg/day, between about 1 g/kg/day and about 100 g/kg/day, between about 2 g/kg/day and about 200 g/kg/day, between about 5 g/kg/day and about 150 g/kg/day, between about 10 g/kg/day and about 100 g/kg/day, between about 5 g/kg/day and about 50 g/kg/day or any intermediate amount or range of PUFA supplement in addition to the PUFA existing in a pet food. A PUFA supplement diet can also include a PUFA level of between about 1 g/1000 kcal and about 10 g/1000 kcal, between about 1 g/1000 kcal and about 5 g/1000 kcal, between about 5 g/1000 kcal and about 10 g/1000 kcal, between about 1 g/1000 kcal and about 3 g/1000 kcal, between about 1 g/1000 kcal and about 2 g/1000 kcal, between about 2 g/1000 kcal and about 4 g/1000 kcal, between about 5 g/1000 kcal and about 8 g/1000 kcal, between about 7 g/1000 kcal and about 10 g/1000 kcal. In certain embodiments, a PUFA supplement diet comprises a PUFA level of about 1 g/1000 kcal, about 2 g/1000 kcal, about 2.1 g/1000 kcal, about 3 g/1000 kcal, about 4 g/1000 kcal, about 5 g/1000 kcal, about 6 g/1000 kcal, about 7 g/1000 kcal, about 8 g/1000 kcal, about 9 g/1000 kcal, or about 10 g/1000 kcal.

In certain non-limiting embodiments, a PUFA supplement diet can include n-6 PUFA, such as plant oils, n-3 PUFA, such as fish oils, eicosapentaenoic acid (EPA), and/or docosahexaenoic acid (DHA).

In certain non-limiting embodiments, an anti-oxidant supplement diet can include between about 0.001% and about 5%, between about 0.01% and about 1%, between about 0.01% and about 2%, between about 0.1% and about 1%, or between about 1% and about 5% anti-oxidant existing in a pet food on a weight by weight basis of a pet food. In some non-limiting embodiments, an anti-oxidant supplement diet comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5% or more anti-oxidant supplement, or any intermediate percentage or range of anti-oxidant supplement, in addition to the anti-oxidant existing in a pet food on a weight by weight basis of a pet food. In other non-limiting embodiments, an anti-oxidant supplement diet can include about 1 mg/kg/day, about 2 mg/kg/day, about 3 mg/kg/day, about 4 mg/kg/day, about 5 mg/kg/day, about 6 mg/kg/day, about 7 mg/kg/day, about 8 mg/kg/day, about 9 mg/kg/day, about 10 mg/kg/day, about 15 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day or more, or any intermediate amount or range of anti-oxidant supplement in addition to the anti-oxidant existing in a pet food. An anti-oxidant supplement diet can include between about 1 mg/kg/day and about 20 mg/kg/day, between about 1 mg/kg/day and about 100 mg/kg/day, between about 2 mg/kg/day and about 200 mg/kg/day, between about 5 mg/kg/day and about 150 mg/kg/day, between about 10 mg/kg/day and about 100 mg/kg/day, between about 5 mg/kg/day and about 50 mg/kg/day or any intermediate amount or range of anti-oxidant supplement in addition to the anti-oxidant existing in a pet food. In certain non-limiting embodiments, the anti-oxidant can be one or more of the following: vitamin E, vitamin C, taurine, carotenoids, flavanols, or any combination thereof. A flavanol, for example, can be catechin, epicatechin, epigallocatechin galate, procyanidins, tannins, or any combination thereof. The anti-oxidant supplement diet can also include a plant that has a high flavanol concentration, such as, cocoa, grapes, and green tea.

In certain non-limiting embodiments, a vitamin B supplement diet can include vitamin B1 (thiamine), vitamin B2 (riboflavin),vitamin B3 (niacin or nicotinamide riboside),vitamin B5 (pantothenic acid),vitamin B6 (pyridoxine, pyridoxal or pyridoxamine),vitamin B7 (biotin),vitamin B9 (folate),vitamin B12 (cobalamins, e.g., cyanocobalamin or methylcobalamin), or any combination thereof. In some non-limiting embodiments, a vitamin B supplement diet can include between about 0.001% and about 2%, between about 0.01% and about 1%, between about 0.05% and about 1%, between about 0.001% and about 0.1%, or between about 0.01% and about 0.2%, vitamin Bs in addition to the vitamin Bs existing in a pet food on a weight by weight basis of a pet food. In other non-limiting embodiments, an vitamin B supplement diet comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2% or more vitamin Bs, or any intermediate percentage or range of vitamin B supplement, in addition to the vitamin Bs existing in a pet food on a weight by weight basis of a pet food. In certain embodiments, a vitamin B supplement diet can include about 1 mg/kg/day, about 2 mg/kg/day, about 3 mg/kg/day, about 4 mg/kg/day, about 5 mg/kg/day, about 6 mg/kg/day, about 7 mg/kg/day, about 8 mg/kg/day, about 9 mg/kg/day, about 10 mg/kg/day, about 15 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day or more, or any intermediate amount or range of vitamin B supplement in addition to the vitamin Bs existing in a pet food. In certain non-limiting embodiments, a vitamin B supplement diet can include between about 1 mg/kg/day and about 20 mg/kg/day, between about 1 mg/kg/day and about 100 mg/kg/day, between about 2 mg/kg/day and about 200 mg/kg/day, between about 5 mg/kg/day and about 150 mg/kg/day, between about 10 mg/kg/day and about 100 mg/kg/day, between about 5 mg/kg/day and about 50 mg/kg/day or any intermediate amount or range of vitamin B supplement in addition to the vitamin Bs existing in a pet food.

In certain non-limiting embodiments, the diet therapy can include one or more of the low phosphorus diet, the calcium supplement diet, the potassium supplement diet, a regular protein diet, or any combination thereof. In some non-limiting embodiments, the diet therapy can include administering to the dog or canine at risk of developing CKD a diet, wherein the diet includes a phosphorus level of about 1.5 g/1000 kcal, a calcium level of about 2 g/1000 kcal, a Ca:P ratio of about 1.3, a potassium level of about 2.1 g/1000 kcal, and a protein level of about 74 g/1000 kcal. In other non-limiting embodiments, the dietary therapy can be any dietary change or therapy known in the art.

In certain non-limiting embodiments, based on the customized recommendation a health practitioner or veterinarian can administer the customized recommendation to the pet or canine.

### 5. Devices, Systems and Applications

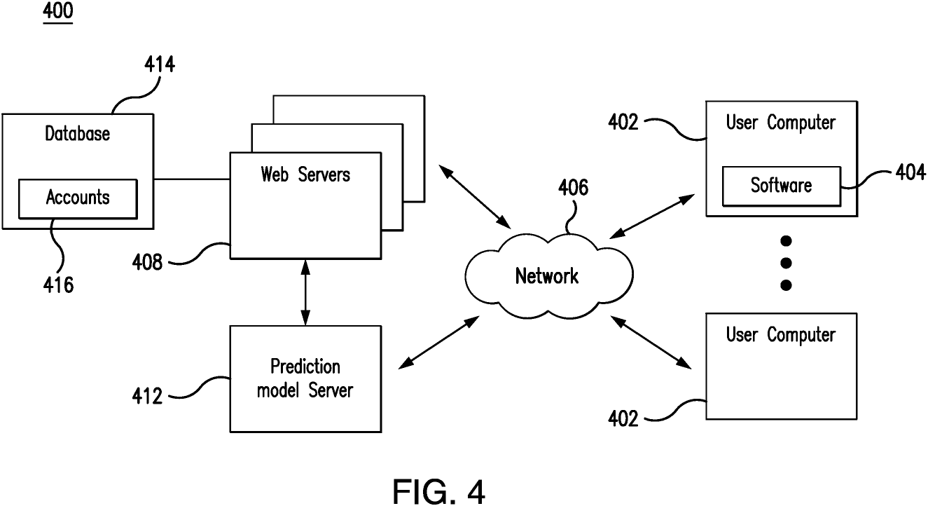

In certain non-limiting embodiments, the embodiments described herein provides a computer system or method for identifying susceptibility of a dog to develop CKD. Any of the steps or processes, including the “receiving,” “processing,” “determining,” or “transmitting,” can be performed by one or more of the devices or apparatuses shown in FIG. 4.

FIG. 4 illustrates a computing system 400 configured for providing a neonatal mortality application in which embodiments of the disclosure can be practiced. As shown, the computing system 400 can include a plurality of web servers 408, prediction model server 412, and a plurality of user computers/equipment (for example, mobile/wireless devices) 402 (only two of which are shown for clarity), each of which can be connected to a communications network 406 (for example, the Internet). The web servers 408 can communicate with the database 414 via a local connection (for example, a Storage Area Network (SAN) or Network Attached Storage (NAS)) over the Internet (for example, a cloud based storage service). The web servers 408 can be configured to either directly access data included in the database 414 or can be configured to interface with a database manager that can be configured to manage data included with the database 414. An account 416 is a data object that can store data associated with a user, such as the user’s email address, password, contact information, billing information, animal information, and the like.

Each user computer 402 can include conventional components of a computing device, for example, a processor, system memory, a hard disk drive, a battery, input devices such as a mouse and a keyboard, and/or output devices such as a monitor or graphical user interface, and/or a combination input/output device such as a touchscreen which not only can receive input but also can display output. Each web server 408 and the prediction model server 412 can include a processor and a system memory (not shown) and can be configured to manage content stored in database 414 using, for example, relational database software and/or a file system. Web servers 408 can be programmed to communicate with one another, user computer 402, and prediction model server 412 using a network protocol such as, for example, the TCP/IP protocol. Prediction model server 412 can communicate directly with the user computer 402, for example, through the communications network 406. The user computer 402 can be programmed to execute software 404, such as web browser programs and other software applications, and can access web pages and/or application managed by web servers 408, for example, by specifying a uniform resource locator (URL) that can direct to web servers 408.

In the embodiments described below, users can respectively operate the user computer 402 that can be connected to the web servers 408 over the communications network 406. Web pages can be displayed to a user via user computer 402. The web pages can be transmitted from the web servers 408 to the user’s computer 402 and can be processed by the web browser program stored in that user’s computer 402 for display through a display device and/or a graphical user interface in communication with the user’s computer 402.

In one example, information and/or images displayed on the user’s computer 402 can relate to customized recommendations or any information included in the health records, including one or more biomarkers and/or demographic information, accessed via an online database. The user’s computer 402 can access the pet’s health information via the communications network 406 which, in turn, retrieves the pet’s health information from the web servers 408 connected to the database 414 and causes the information and/or images to be displayed through a graphical user interface of the user’s computer 402. The online information and/or images, and/or the neonatal mortality application, can be managed with a username and password combination, or other similar restricted access/verification required access method, which can allow the user to “log in” and access the information.

It is noted that the user computer 402 can be a personal computer, laptop, mobile computing device, smart phone, video game console, home digital media player, network-connected television, set top box, and/or other computing devices having components suitable for communicating with the communications network 406. The user computer 402 can also execute other software applications configured to receive a customized recommendation from a prediction model server, such as, but not limited to, text and/or image display software, media players, computer and video games, and/or widget platforms, among others.

FIG. 5 illustrates a more detailed view of the prediction model server 412 of FIG. 4. The prediction model server 512 can include, without limitation, a central processing unit (CPU) 502, a network interface 504, memory 520, and storage 530 communicating via an interconnect 506. The prediction model server 512 can also include I/O device interfaces 508 connecting I/O devices 510 (for example, keyboard, video, mouse, audio, touchscreen, etc.). The prediction model server 512 can further include the network interface 504 configured to transmit data via data communications network 406.

CPU 502 can retrieve and execute programming instructions stored in the memory 520 and can generally control and coordinate operations of other system components. Similarly, the CPU 502 can store and retrieve application data residing in the memory 520. The CPU 502 can be included to be representative of a single CPU, multiple CPUs, a single CPU having multiple processing cores, and the like. The interconnect 506 can be used to transmit programming instructions and application data between CPU 502, I/O device interfaces 508, storage 530, network interfaces 504, and memory 520.

Memory 520 can be generally included to be representative of a random access memory and, in operation, stores software application and data for use by the CPU 502. Although shown as a single unit, the storage 530 can be a combination of fixed and/or removable storage devices, such as fixed disk drives, floppy disk drives, random access memory, hard disk drives, non-transitory computer-readable medium, flash memory storage drives, tape drives, removable memory cards, CD-ROM, DVD-ROM, Blu-Ray, HD-DVD, optical storage, network attached storage (NAS), cloud storage, or a storage area-network (SAN) configured to store non-volatile data.

Memory 520 can store instructions and logic for executing an application platform 526 which can include images 528 and/or prediction model software 538. Storage 530 can store images and/or information 534 and other user generated media and can include a database 532 which can be configured to store images and/or information 534 associated with the application platform content 5236. The database 532 can be any type of storage device, and/or can include one or more datasets described herein. Database 532 can store application content relating to data associated with user generated media or images. Database 532 can also include one or more biomarkers or demographic information, and/or customized recommendations.

Network computers are another type of computer system that can be used in conjunction with the disclosures provided herein. Network computers do not usually include a hard disk or other mass storage, and the executable programs can be loaded from a network connection into the memory 520 for execution by the CPU 502. A web TV system can be also considered to be a computer system, but it can lack some of the features shown in FIG. 5, such as certain input or output devices. A typical computer system will usually include at least a processor, memory, and an interconnect coupling the memory to the processor.

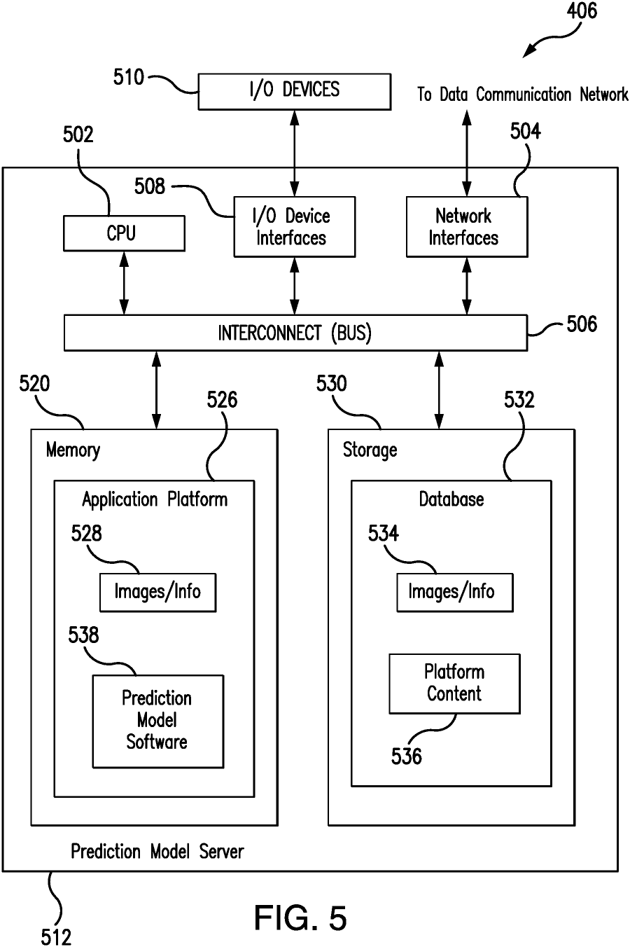

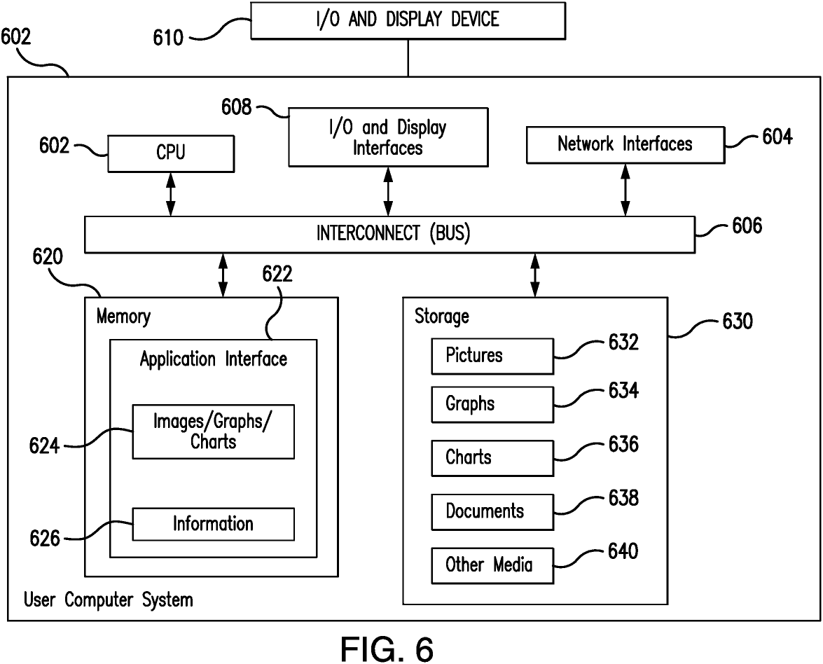

FIG. 6 illustrates a user computer or equipment 402 used to access the prediction model server 412 and display images and/or information associated with the application platform 620. User computer or user equipment 602, for example, can be a desktop computer, a laptop computer, a mobile device, or any other user equipment. User computer 602 can include, without limitation, a central processing unit (CPU) 602, a network interface 604, an interconnect 606, a memory 620, and storage 630. User computer 602 can also include an I/O device interface 608 connecting I/O devices 610 (for example, keyboard, display, touchscreen, and mouse devices) to the user computer 602.

Like CPU 502, CPU 602 can be included to be representative of a single CPU, multiple CPUs, a single CPU having multiple processing cores, etc., and the memory 620 can be generally included to be representative of a random access memory. Interconnect 606 can be used to transmit programming instructions application data between the CPU 602, I/O device interfaces 608, storage 630, network interface 604, and memory 6320. Network interface 604 can be configured to transmit data via the communications network 406, for example, to stream or provide content from the prediction model server 512. Storage 630, such as a hard disk drive or solid-state storage drive (SSD), can store non-volatile data. Storage 630 can contain pictures 632, graphs 634, charts 636, documents 638, and other media 640. Illustratively, the memory 620 can include an application interface 622, which itself can display images 624, such as graphs or charts among others, and/or information 626. The application interface 622 can provide one or more software applications which can allow the user to access media items and other content hosted by the prediction model server 412.

All of the above terms are merely convenient labels applied to these physical quantities. Unless specifically stated otherwise as apparent from the following discussion, it is appreciated that throughout the description, discussions utilizing terms such as “processing” or “computing” or “calculating” or “determining” or “displaying” or “analyzing” or the like, refer to the action and processes of a computer system, server, or any other electronic computing device, that manipulates and transforms data represented as physical (electronic) quantities within the computer system’s registers and memories into other data, similarly represented as physical quantities within the computer system memories, registers, or other such information storage, transmission, or display devices.

The present example also relates to an apparatus for performing the operations herein. This apparatus can be specially constructed for the required purposes, or it can comprise a general purpose computer selectively activated or reconfigured by a computer program stored in the computer. Such a computer program can be stored in a computer readable storage medium, such as, but is not limited to, read-only memories (ROMs), random access memories (RAMs), EPROMs, EEPROMs, flash memory, magnetic or optical cards, any type of disk including floppy disks, optical disks, CD-ROMs, and magnetic-optical disks, or any type of media suitable for storing electronic instructions, and each coupled to a computer system interconnect.

The algorithms and displays presented herein are not inherently related to any particular computer or other apparatus. Various general-purpose systems can be used with programs in accordance with the teachings herein, or it can prove convenient to construct a more specialized apparatus to perform the required method operations. The structure for a variety of these systems will appear from the description above. In addition, the present examples are not described with reference to any particular programming language, and various examples can thus be implemented using a variety of programming languages.

The algorithms and displays presented herein are not inherently related to any particular computer or other device. Various general-purpose systems may be used with the application in accordance with the teachings herein, or it may prove convenient to construct a more specialized device to perform the required method operations. The structure for a variety of these systems will appear from the description above. In addition, the present embodiments are not described with reference to any particular programming language, and various examples may thus be implemented using a variety of programming languages. All preferred features and/or embodiments of the methods and the diets/dietary regimes disclosed in the instant application apply to the device, the system and the application.

### EXAMPLES

The presently disclosed subject matter can be better understood by reference to the following. The below examples are exemplary only and should in no way be taken as limiting.

### Example 1

To determine performance of the CKD prediction model around the time of diagnosis, the prediction model was applied to 15,044 of 18,705 dogs or canines in the testing dataset that had a visit 3 months or less before the time of evaluation T(0). The prediction model showed a sensitivity of 91.4% (687/752) for those dogs or canines classified as “CKD” and a specificity of 97.2% (13891/14292) for those dogs or canines classified as “no CKD”. Given that age is a feature of the prediction model, sensitivity and specificity were also reported by age at evaluation (T0).

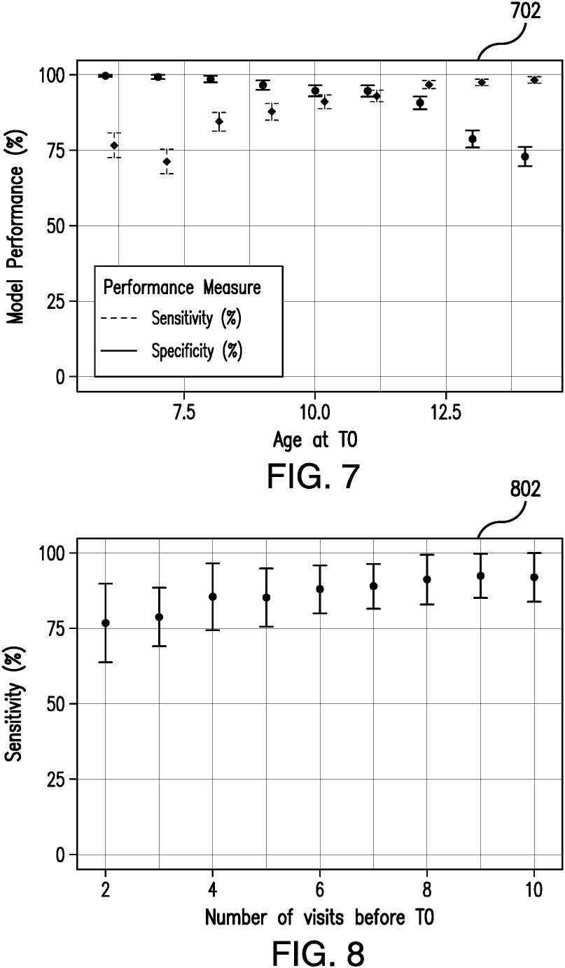

FIG. 7 illustrates a graph 702 showing performance metrics according to certain embodiments described herein. Specificity, for example, was consistently above 98% until an age of 8 years and declined thereafter reaching 67.0% for an age of 15 years. Sensitivity, on the other hand, increased with age, and is over 96% from 12 years old onwards. The prediction model can sacrifice some specificity (increased false positive rate) for better sensitivity (lower false negative rate) when predicting older pets, in which CKD prevalence is much higher, hence optimising overall accuracy for each age group.

FIG. 8 illustrates a graph 802 showing performance metrics according to certain embodiments described herein. To understand how the patient history affected model performance, model sensitivity can be examined as a function of the number of visits in the health records before the diagnosis was made. Sensitivity can increase from 76.8% with 2 visits, to 85.5% with 4 visits prior to the diagnosis, and continues to improve to over 92% with further data. This illustrates that historical information can contribute to the quality of the CKD diagnosis.

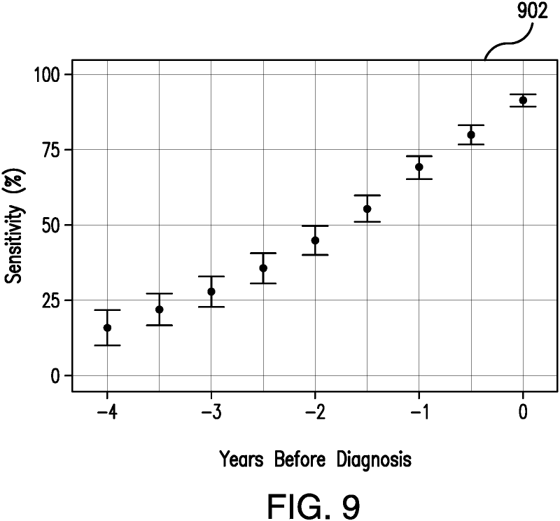

FIG. 9 illustrates a graph 902 showing performance metrics according to certain embodiments described herein. As the CKD diagnosis benefits from historic health record information, the model’s ability to predict a future CKD diagnosis can be evaluated. To evaluate the prediction model, the health records can be truncated for “CKD” at different time points before diagnosis. For example, in a 1-year early prediction all information between the diagnosis and 1 year before can be removed. The ability of the prediction model to predict future onset of CKD can then be evaluated. As expected, sensitivity decreased when increasing the time between prediction and diagnosis, although of the dogs that went on to develop CKD 69.1% were correctly predicted 1 year before diagnosis, 44.9% 2 years before diagnosis, and 22.0% as far as 3.5 years prior to first or original CKD diagnosis.

In certain non-limiting embodiments, assessing specificity by truncating the dataset may not make sense, given that as dogs remain classified as “no CKD” at all earlier visits. The specificity for early CKD detection can be best appreciated by its distribution with age, as shown in FIG. 7, where the average level can be above 90% up to a dog age of 12.

In some non-limiting embodiments, advanced computational modelling can be used to predict CKD risk based on current and/or past health records. This dataset can include clinicopathologic results, which were evaluated and refined. The prediction model can include one or more of the following six features: serum creatinine, blood urea nitrogen, urine protein and urine specific gravity, and age and weight of the patient. The prediction model can indicate a sensitivity of 76.8% with two visits, increasing to 85.5% with four visits, which emphasizes the value of regular proactive health monitoring and in obtaining complete clinical pathology data whenever possible. Specificity can continue to increase over 92% with additional data. In some non-limiting embodiments, specificity, indicated by a low false positive rate, can be helpful, especially when considering predictive testing and subsequent clinical decision making and owner communications.

The current prediction model, such as a 5-3-3 RNN, can differ from previously described prediction model, such as a 7-3 RNN, in that it includes one or more of urine protein, patient weight, or any other additional biomarker or demographic information. In certain non-limiting embodiments, renal protein loss and resultant proteinuria can be a more common feature in canine renal disease than in dog or canine renal disease. Patient weight was determined to be a helpful component of the prediction model, likely due to the larger range of weights that may be seen in canine patients, with some variation in disease prevalence apparent between dogs of different sizes. In certain non-limiting embodiments, both challenges in maintaining adequate nutrition and lean body mass, as well as difficulties in providing the requisite nursing care to patients with CKD, may also negatively impact prognosis and outcome in larger patients.

In some non-limiting embodiments, an individual patient’s clinical pathologic variables can vary to a degree over time and/or can influence by factors such as changes in dietary intake, posture, muscle mass, and/or hydration status. The reference intervals, which are commonly utilized to evaluate clinical pathology results, can provide a limited interpretation and may not accurately reflect an individual patient’s unique status. Using prediction modelling can help to accurately diagnose CKD.

### Example 2

In certain non-limiting embodiments, prediction model can include an RNN. The RNN include a 5-3-3 architecture, 10-fold, and 18 epochs. The RNN would evaluate 7 selected features, such as one or more biomarkers and/or demographic information. For example, the 7 selected features can be BUN, urine sg, visiting age, creatinine, urine protein, weight, and/or amylase. Performance of the RNN was measured for pets having CKD at a random, stratified point with a two-year period. The prediction model achieved a 94.2% area under the receiver operating characteristics (AUCROC), a 91.6% area under the precision-recall curve (AUCPR), and an 82.6% F1-score.

In some non-limiting embodiments, the dataset used to train the prediction model included about 306,757 visit records for about 39,442 unique dogs or canines. Of the 39,442 unique dogs or canines, about 26,514 had no CKD and about 12,928 either had or developed CKD. The dataset included 35 features, including demographic information and one or more biomarkers drawn from blood chemistry, haematology, and/or urine levels. Examples of the one or more biomarker can include, but are not limited to, alkaline phosphatase, amylase, protein, BUN or urea level, creatinine, phosphorus, calcium, urine protein, potassium, glucose, hematocrit, hemoglobin, red blood cell (RBC) count, red cell distribution width (RDW), alanine aminotransferase, albumin, bilirubin, chloride, cholesterol, eosinophil, globulin, lymphocyte, monocyte, mean corpuscular hemoglobin (MCH), mean corpuscular hemoglobin concentration (MCHC), mean corpuscular volume (MCV), mean platelet volume (MPV), platelet count, segmented neutrophils, sodium, urine PH level, and/or white blood cell count. The demographic information can include, for example, the age and weight of the dog or canine, as well as the age in which the dog or canine was first or originally diagnosed with CKD.

The number of visits per dog or canine included in dataset ranges from 1 to 40. For example, the average number of visits per dog or canine can be 7.7. The number of visits can be skewed towards 1 to 15 visits, with the number of visits rapidly increasing from 1 to 4 visits, and then steadily decreases from 5 to 40 visits. In certain non-limiting embodiments, the dataset can include missing information for one or more of the 35 features. For example, the features with missing values greater than 60% include urine protein, about 66.5%, urine specific gravity, about 64.2%, potassium, about 70%, chloride, about 70.4%, eosinophil, about 60.6%, sodium, about 70.2%, and urine pH, about 64.8%. Any other feature, such as the one or more biomarkers or demographic information, can be missing between 0% to 100% of the data from the database. The missing information can be imputed.

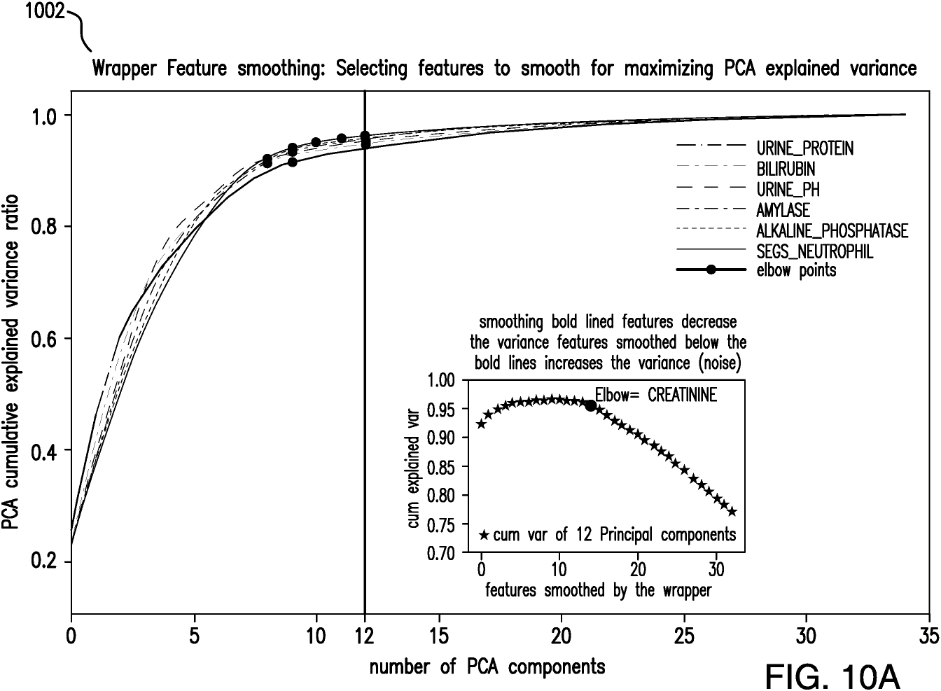

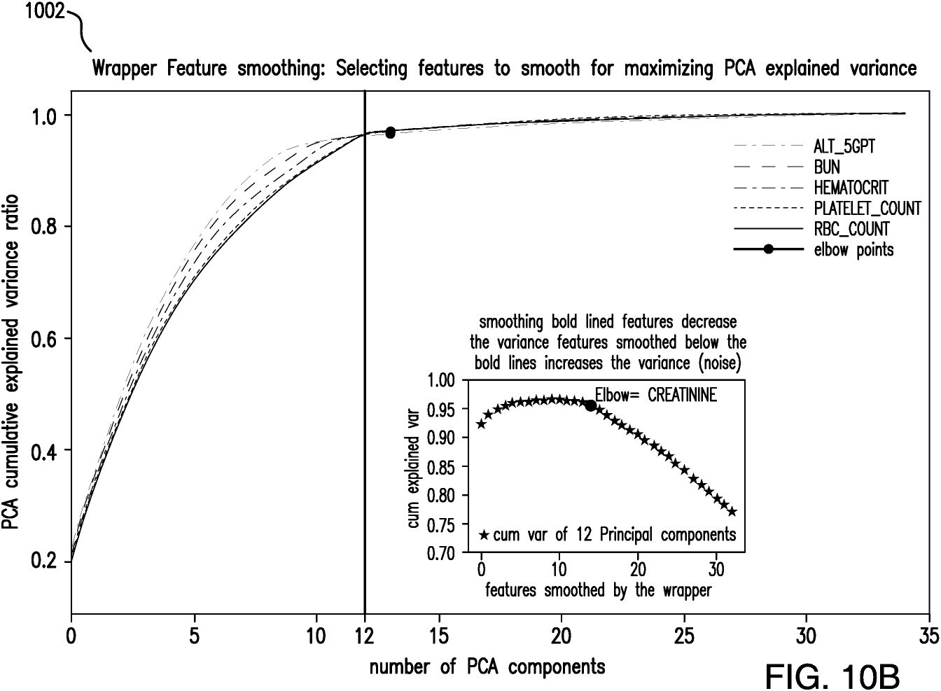

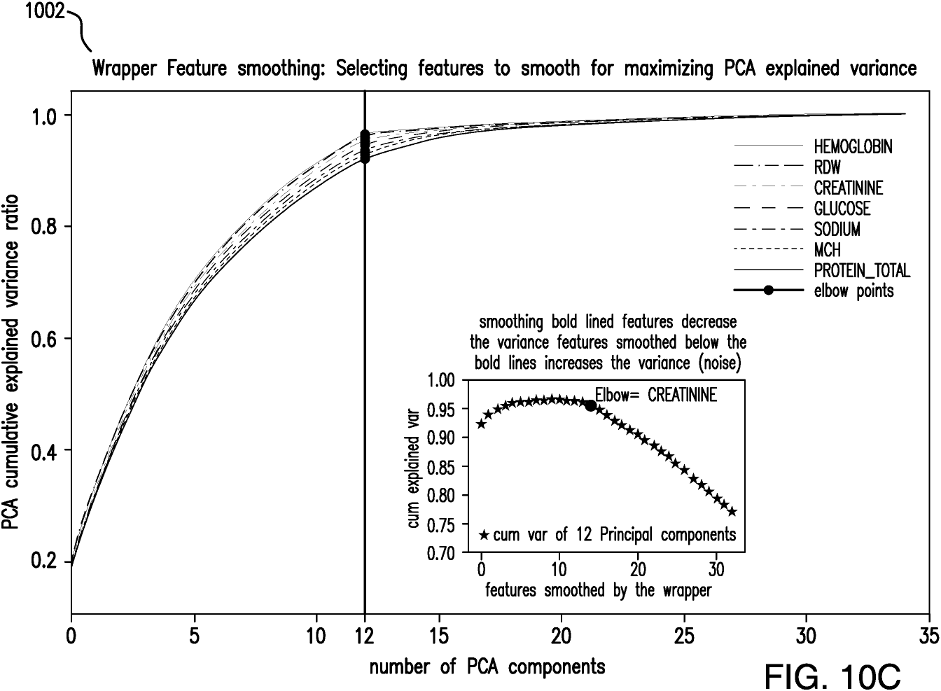

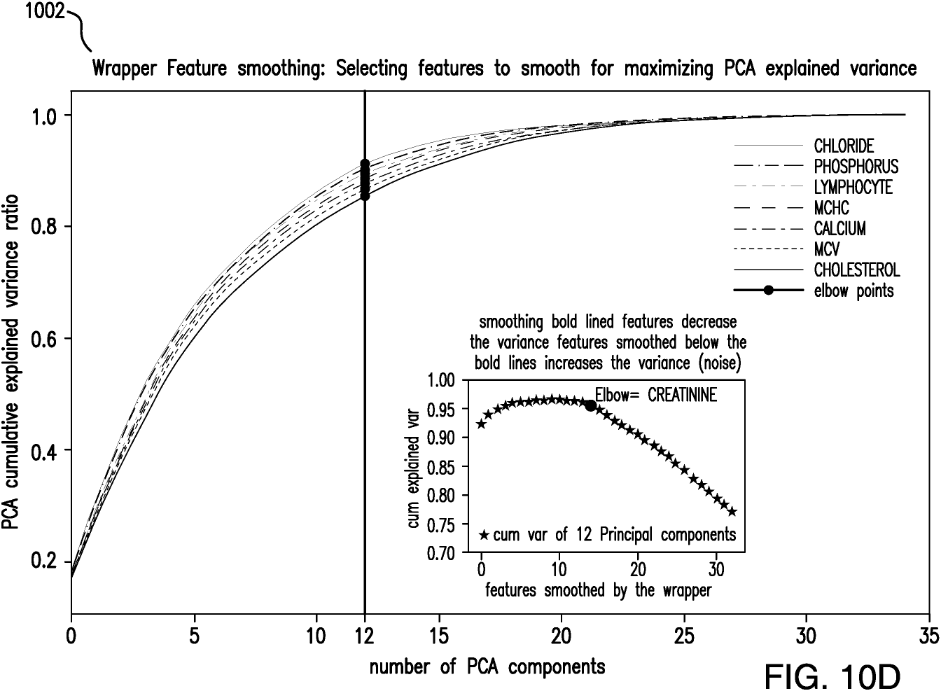

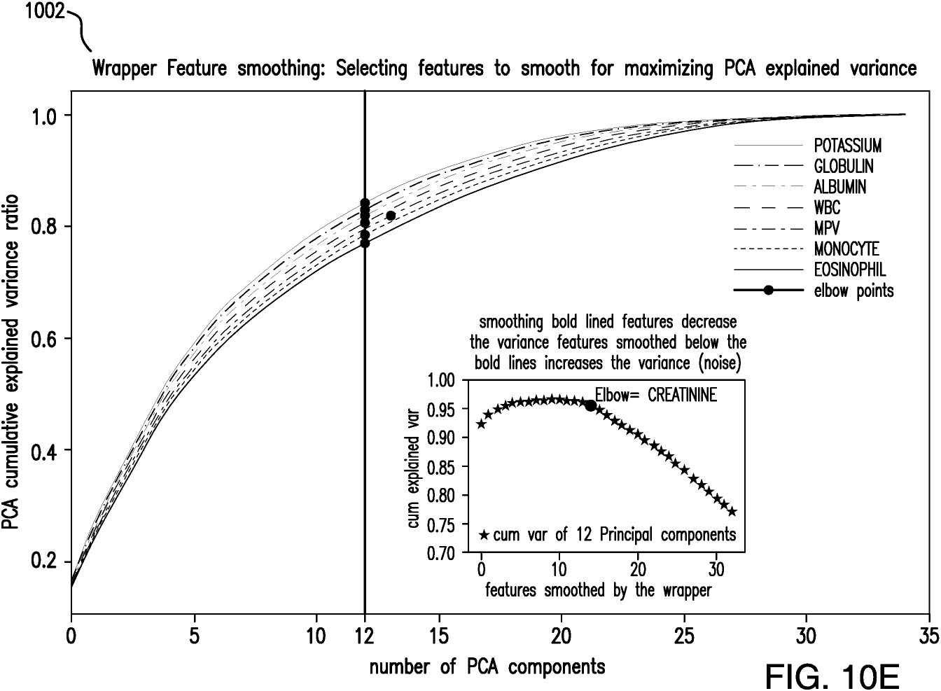

FIGS. 10A - 10E illustrate pre-processing of the dataset 1002 according to certain embodiments described herein. For example, as part of the pre-processing the missing data within the database can be imputed using, for example, a random forest implementation. In another example, the data can undergo a min-max normalization for each feature into a range between 0 and 1. In yet another example, the pre-processing can include a pre-smoothing feature by thresholding their values to predefine min-max limits. As shown in FIGS. 10A - 10E, a bottom-up wrapper can be employed that selects which feature to smooth for maximizing the cumulative explained variance as computed, for example, via principal component analysis (PCA). Accordingly, the values shown in FIGS. 10A - 10E range from 0 to 1 and are smoothed for maximining PCA explained variance. When all features are smoothed, the explained variance can be 75%, which indicates a noisy dataset. By optimizing feature smoothing, the PCA explained variance can become 95%.

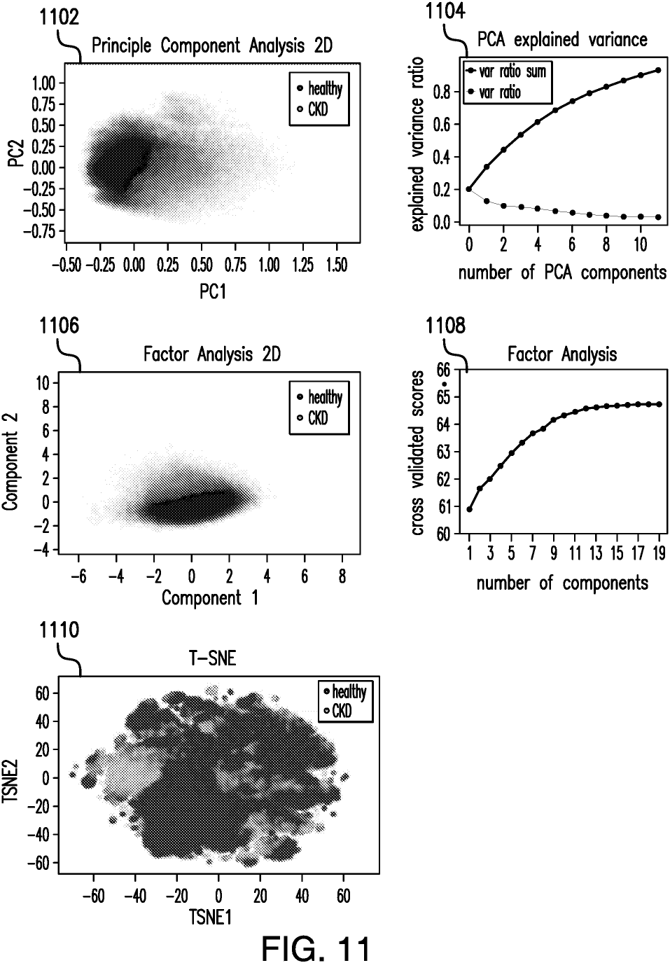

FIG. 11 illustrates principal component analysis or factor analysis according to certain embodiments described herein. For example, graph 1102 illustrates a projection of the dataset into a two-dimensional space by performing a 2-D PCA linear. Graph 1104, on the other hand, shows the PCA explained variance and the variance ration, which can confirm that the dataset is not noisy. In some non-limiting embodiments, graph 1108 can illustrate a factor analysis in two dimensions, measuring the number of components against the cross-validation scores. Graph 1106 can illustrate factor analysis in two dimensions. Graph 1110 can illustrate the t-Distributed Stochastic Neighbor Embedding (t-SNE) non-linear dimensionality reduction in two dimensions.

After the dataset is pre-processed, a kernel density estimation can be used on each feature for finding the normalized histograms that can be used to compute the true probability density function (PDF) at each bin, and/or illustrate the underlying distribution from which the data was originally sampled. After analysis of the data using PDF, in certain non-limiting embodiments the most discriminative features can be BUN, urine specific gravity, creatinine, and visiting ages. For each of these features, the CKD/healthy normalized distributions were found to be different in their regions and range. Other features, such as amylase, urine protein, and cholesterol also appeared to be distinctive feature that could be useful.

In certain non-limiting embodiments univariate feature ranking was employed on the pre-processed dataset. The ranking of the features, for example, included a fast filter method based on signal-to-noise ratio (SNR), including a first order and/or a second order type, as well as embedded machine learning methods. The results of the fast filter method and/or the machine learning methods can be combined to create average rankings of the features. The fast filter methods, for example, can include one or more of the following: correlation coefficient f-score, class conditional SNR, two sample test statistic, symmetric divergence, and/or fisher discriminant ratio. The method learning method, for example, can include one or more of the following: randomized lasso, ridge regression, random forest, and/or recursive feature elimination. Based on the normalized weights of one or more of the above fast filter and/or machine learning methods, the top four features can be visiting age, BUN, urine specific gravity, and creatinine. The next three features were amylase, cholesterol, and/or urine protein. The remaining features were ranked as follows: potassium, alkaline phosphatase, lymphocyte, MCV, weight, globulin, urine PH, haematocrit, phosphorus, platelet count, haemoglobin, alanine aminotransferase, RDW, MCH, chloride, segmented neutrophils, RBC count, MPV, total protein, white blood cell count, glucose, calcium, MCHC, albumin, sodium, eosinophil, monocyte, and/or bilirubin.

In some non-limiting embodiments, the prediction model can be designed to determine whether a given canine or dog will develop CKD within the next two years. To design a prediction model to accurately address the above, a pan-pet database can be construction, which can be a superset of all possible visit trajectories. For a dog or canine with N visits, the trajectory can be defined temporally according to the list of visits. A reduced trajectory can then be ordered having any ordered subset of visits, with the last K visits being removed. K can be any number of visits between 1 and N. When the original dataset is extended to include all possible reduced trajectories for CKD, with removed visits up to 2 years before diagnosis, the resulting augmented dataset can be referred to as a pan-canine dataset.

The samples dataset, in certain non-limiting embodiments, can be the subset of pan-canine dataset in which a single trajectory for each CKD dog or canine is selected. A large number of sampled datasets can be created using a random number generator with different seeds, so that a different trajectory (e.g., a different number of visits) can be chosen for each pet identification (e.g., sampling with replacement). When using a sampled dataset, a predictor can learn the patterns for pets that will be diagnosed with CKS at any point in the next 2 years.

In certain non-limiting embodiments, a RNN with a 3-7 configuration can be used to produce baseline performances of the prediction model using the dataset. For example, the RNN with a 3-7 configuration can be trained with 10-fold cross validation and 16 epochs. This RNN can then be used to show the performance of the four top features, which can be visiting age, BUN, creatinine, and/or urine specific gravity, as well as performance of all 35 features. The performance of the four top features using the RNN with a 3-7 configuration yielded a sensitivity of 76.2%, a specificity of 93.9%, an accuracy of 88.2%, an F1 score of 80.6%. On the other hand, performance of all 35 features using the RNN with a 3-7 configuration yielded a sensitivity of 77.2%, a specificity of 95.1%, an accuracy of 89.3%, an F1 score of 82.3%.

To reduce total computation costs by a factor of six, the RNN 3-7 configuration can be trained with 3-fold cross validation and 10 epochs. This RNN can then be used to show the performance of the four best features, which can be visiting age, BUN, creatinine, and/or urine specific gravity, as well as performance of all 35 features. The performance of the four top features using this RNN yielded a sensitivity of 76.3%, a specificity of 92.5%, an accuracy of 87.3%, an F1 score of 79.5%. On the other hand, performance of all 35 features using the RNN with a 3-7 configuration yielded a sensitivity of 75.1%, a specificity of 95.0%, an accuracy of 88.6%, an F1 score of 80.9%.

Another comparison can be using RNN with LSTM (LTSM 3-7) trained with 10-fold cross validation and 16 epochs. This LSTM 3-7 can then be used to show the performance of the four top features, which can be visiting age, BUN, creatinine, and/or urine specific gravity. The performance of the four top features using LTSM 3-7 yielded a sensitivity of 75.7%, a specificity of 94.3%, an accuracy of 88.3%, an F1 score of 80.7%. A further comparison can be using an RNN with a 3-5-3 configuration, trained with a 10-fold cross validation and 20 epochs using the six top features, such as BUN, creatinine, urine protein, urine specific gravity, urine PH, and WBC. The performance of the six top features using RNN with a 3-5-3 configuration yielded a sensitivity of 75.9%, a specificity of 93.0%, an accuracy of 87.5%, an F1 score of 79.6%.

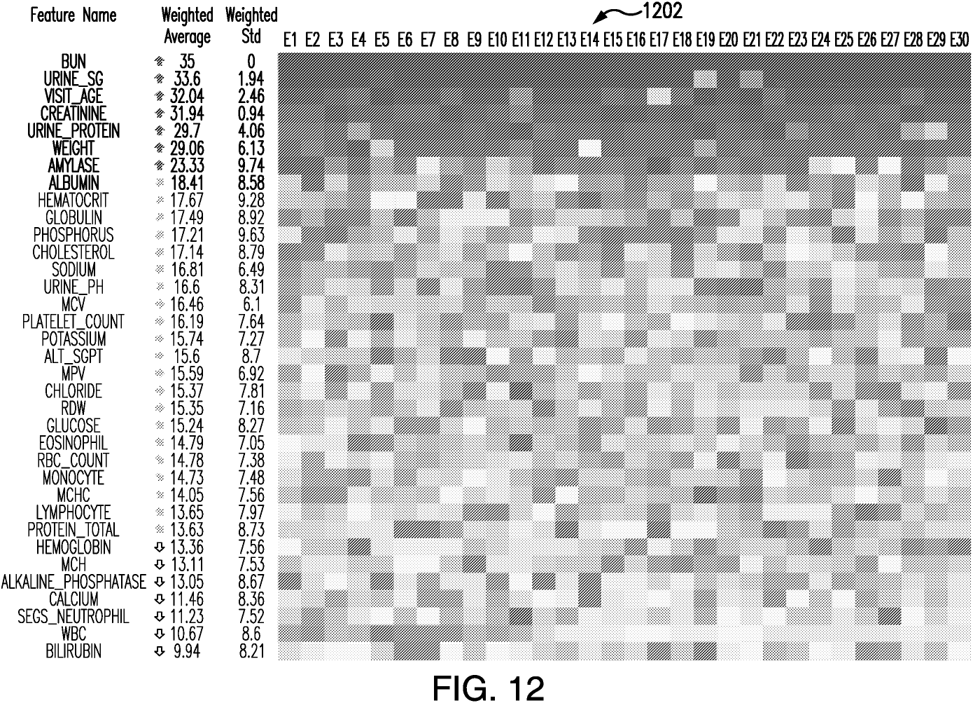

FIG. 12 illustrates a wrapper-based feature ordering chart 1202 according to certain embodiments described herein. In certain non-limiting embodiments supervised feature selection can be conducted using a top-down wrapper or a bottom-up wrapper method. In other embodiments supervised feature selection can be conducted using both a top-down wrapper and a bottom-up wrapper method. The prediction model, for example, can be a RNN with a 3-7 architecture, trained using 3-fold cross-validation and 10 training epochs. For example, several top-down and bottom-down wrapper feature selection experiments can be conducted by changing the bootstrap samples and randomness inside the RNN cross-validation. As shown in FIG. 12, the results can be assembled to create average wrapper-based feature selection. In particular, the average wrapper-based feature orderings via the average position (POS) sorted from the top selected features to the least selected. In some non-limiting embodiments 30 different experiments with different randomness can be included. The arrows shown in FIG. 12 illustrate the quantiles with respect to the selected order of the feature from each different wrapper experiment. As shown in FIG. 12, the top seven features can be BUN, urine specific gravity, visit age, creatinine, urine protein, weight, and/or amylase.

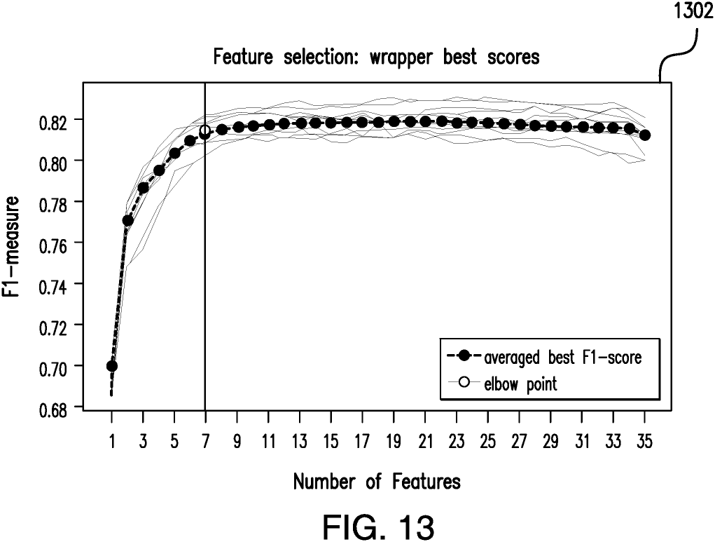

FIG. 13 illustrates the averaged best F1-scores 1302 according to certain embodiments described herein. In certain non-limiting embodiments, the averaged best F1-scores of the several bootstrap sampling experiments with top-down and bottom-up wrapper method can be charted. As shown in FIG. 13, the F1-scores can start dropping after keeping less than seven features. Therefore, the prediction model can utilize seven features, including a combination of one or more biomarkers and/or demographic information. The seven features, for example, can be BUN, urine specific gravity, visit age, creatinine, urine protein, weight, and/or amylase.

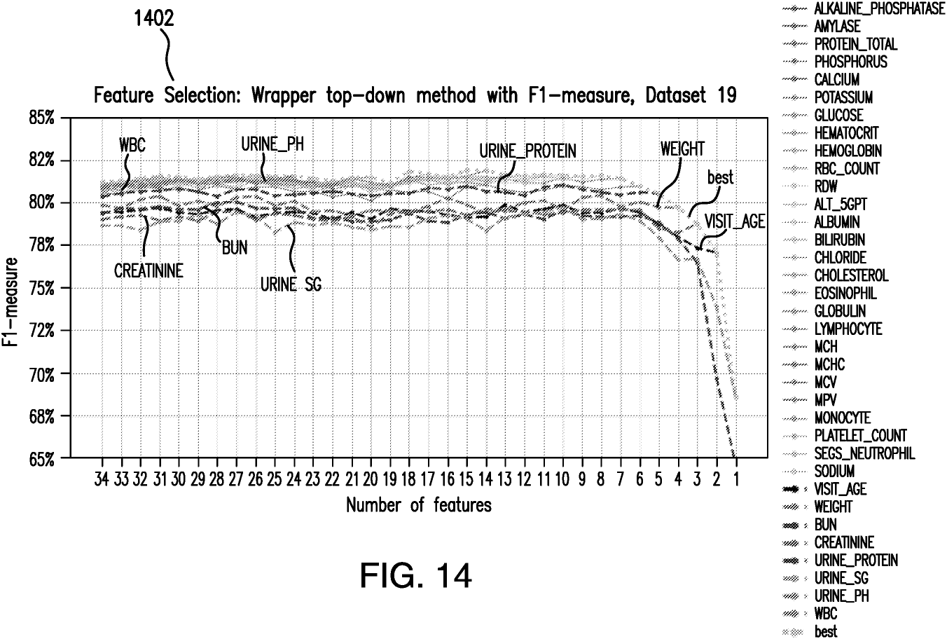

FIG. 14 illustrates a chart 1402 showing feature selection according to certain embodiments described herein. In particular, FIG. 14 illustrates a feature selection with top-down wrapper method via the RNN predictor. As shown in FIG. 14, the top seven selected featured include BUN, urine specific gravity, visit age, creatinine, urine protein, weight, and/or amylase.

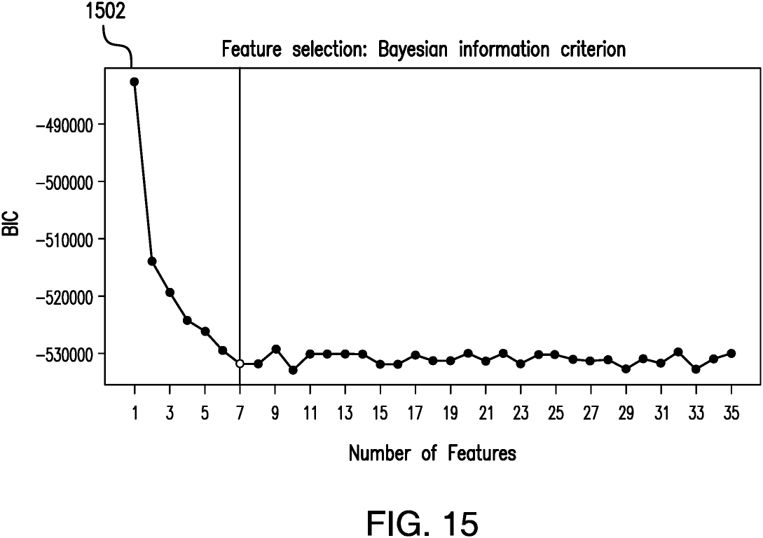

FIG. 15 illustrates a graph 1502 showing Bayesian information criterion during wrapper feature selection according to certain embodiments described herein. In particular, FIG. 15 illustrates a Bayesian information criterion of the dataset feature selection illustrated in FIG. 14. As shown in FIG. 15, the knee point corresponding to the point where seven features are selected. For example, the seven features can include BUN, urine specific gravity, visit age, creatinine, urine protein, weight, and/or amylase.

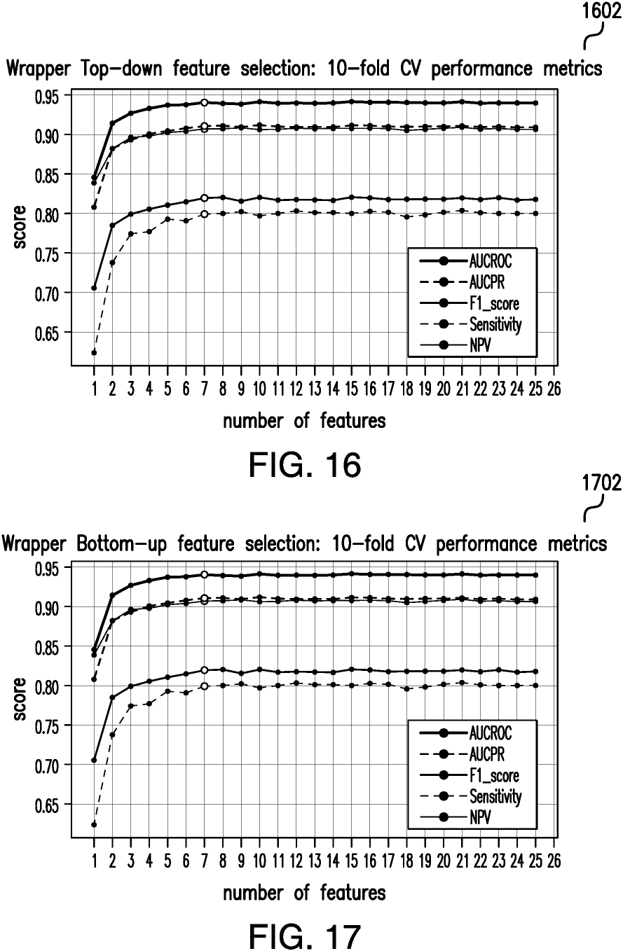

FIG. 16 illustrates a graph 1602 showing performance metrics according to certain embodiments described herein. Specifically, FIG. 16 illustrates a wrapper top-down feature selection, including a 10-fold cross validation performance metric. This metric allowed for optimizing F1-measure, via grid search over different thresholds, at one or more of the steps. Performance metrics, such as AUCPR, AUCROC, sensitivity, or NPV can be calculated during the wrapper feature selection process. FIG. 16 illustrates that the optimal number of selected features can be seven.

FIG. 17 illustrates a graph 1702 showing performance metrics according to certain embodiments described herein. Specifically, FIG. 17 illustrates a wrapper bottom-up feature selection, including a 10-fold cross validation performance metric. This metric allowed for optimizing F1-measure, via grid search over different thresholds, at one or more of the steps. Performance metrics, such as AUCPR, AUCROC, sensitivity, or NPV can be calculated during the wrapper feature selection process. Similar to FIG. 16, FIG. 17 illustrates that the optimal number of selected features can be seven.

In certain non-limiting embodiments, the RNN architecture can be optimized. For example, for RNN-LSTM, different configurations of 1-5 hidden layers and 3-200 nodes per layer can be tested. TanH activation function can be used in the hidden layers, with softmax being used at the output layer. The softmax can be sigmoid, given the binary classification of no CKD or CKD. Binary cross-entropy can also be used for loss calculation, and/or a 20% dropout can be considered to avoid overfitting. Backpropagation through time can be used for training with the RMSprop gradient descent optimization algorithm. Further, in some other non-limiting embodiments LSTM cell structure can be tested to cope with vanishing gradients.

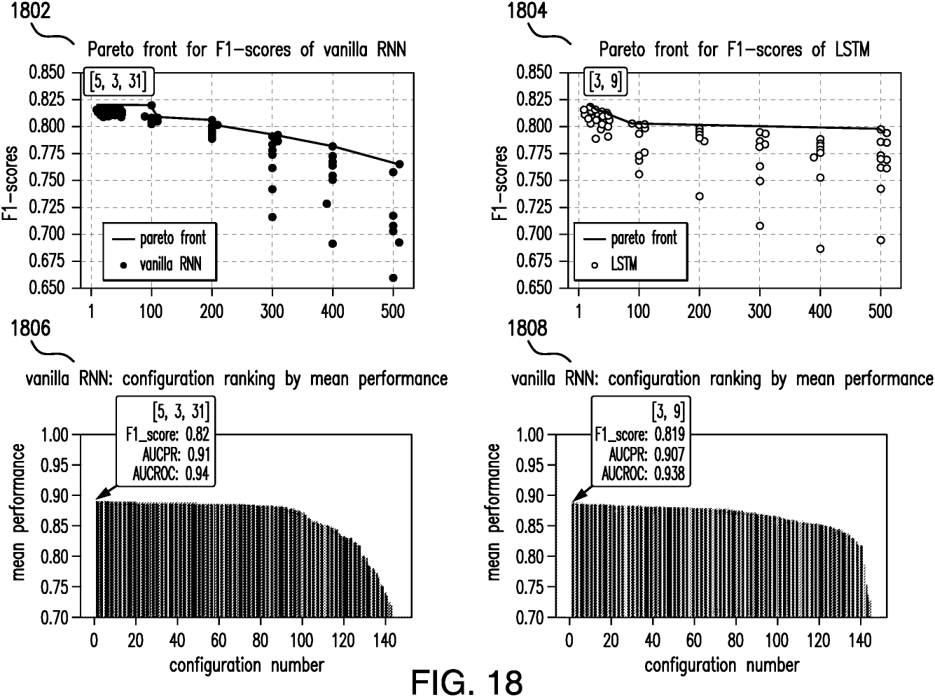

FIG. 18 illustrates a graph showing a RNN and LSTM architectures according to certain embodiments described herein. As shown in FIG. 10, the F1 measure or score changes as a function of the total number of nodes. The top performing configurations after a 10-fold cross validation, for example, were a 3-layer Vanilla RNN (5-3-3) and/or a 2-layer RNN-LSTM with a 3-9 architecture. Pareto front can then be used to find the optimums for both F1-score and the number of nodes or neurons in the RNN. As shown in graph 1802, the best performing RNN can include a 5-3-3 architecture, while in graph 1804 the best performing LSTM can include a 3-9 architecture. As shown in graph 1806, the RNN or vanilla RNN with 5-3-3 architecture had an F1 score of 0.82, an AUCPR of 0.91, and an AUCROC of 0.94. On the other hand, the LSTM with 3-9 architecture had an F1 score of 0.819, an AUCPR of 0.907, and an AUCROC of 0.938, as shown in graph 1808.

Other vanilla RNN were tested including, but is not limited to, 5-3-3, 9-3-0, 5-5-0, 3-7-0, 5-5-10, 7-3-0, 8-4-0, 3-9-0, 7-5-2, 20-0-0, 30-0-0, 2-6-3, 5-9-0, 4-2-4, 5-5-5-5, 3-3-3-3, 3-3-3, 7-9-4-8, 5-4-6-3, 4-8-4, 9-3-6-9. As described above, RNNs with 1-5 layers, with each layer having anywhere between about 1 and about 250 nodes were tested. While an RNN with an architecture of 5-3-3 was chosen, in certain other embodiments any other RNN can be selected.

In addition, other LSTMs were tested including, but is not limited to, 3-9-0, 5-5-0, 3-3-3, 7-3-0, 2-4-4, 2-6-3, 4-8-4, 3-3-5, 7-13-0, 3-3-3-3, 6-4-6, 3-7-0, 9-3-0, 10-10-0, 5-9-0, 8-4-0, 7-3-7-3, 8-4-8, 20-20-0, 3-9-6-5, 5-3-3-5-6. As described above, LSTMs with 1-5 layers, with each layer having anywhere between about 1 and about 250 nodes were tested. While an LSTMs with an architecture of 3-9-0 was chosen, in certain other embodiments any other LSTM can be selected.

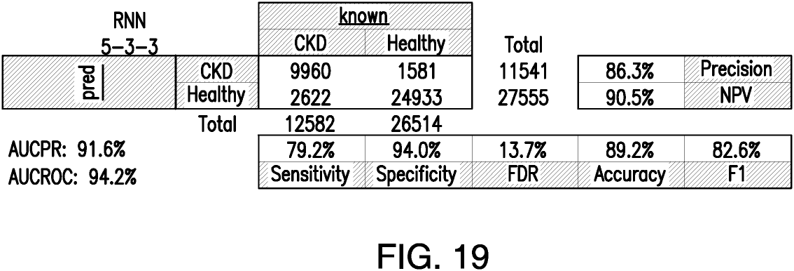

FIG. 19 illustrates cross-validation performance 1902 according to certain embodiments described herein. In particular, an RNN with a 5-3-3 architecture can be used. The RNN can include 10 folds, and 18 epochs. The RNN can yield a sensitivity of 79.2%, a specificity of 94%, an accuracy of 89.2%, and an F1 score of 82.6%.

In certain non-limiting embodiments, temperature scaling can be used. Due to the nature of the RNN outputs, which uses a softmax or sigmoid function, probabilities can be re-calibrated as they occupy neighborhoods close to the boundary between 0 and 1. Temperature scaling, for example, can be used as a single parameter variant of Platt Scaling. The temperature scaling parameter can be determined by minimizing the negative log likelihood (e.g., cross entropy loss) using the following equation: sigmoid(logit(pᵢ)/T), where pᵢ equals the initial neural network prediction for each i pet. In some non-limiting embodiments, the chosen temperature parameter T was found equal to 1.297 using a 10-fold cross validation of the model.

In certain non-limiting embodiments, the prevalence of CKD in the dataset was determined to be 33%. The decision threshold can be re-optimized for 5% prevalence, which can be a bit higher than for most breeds, but representative of senior dogs, given that current diagnosis may miss 50% of ‘at risk’ pets. The prediction model, in some non-limiting embodiments, is not re-trained by using a dataset of 5% CKD class proportion. Rather, the decision threshold of the prediction model can be chosen for a 5% prevalence.

The decision threshold calculation can be performed via 100 iterations of under-sampling the CKD class calibrated prediction probabilities, for example, random sampling of 1400 disease dog or canines gives 5%. The averaged performance metrics during grid searching decision thresholds range from 0.05 to 0.95 with step of 0.05. This chosen decision threshold can depend on the metric chosen to evaluate the threshold. For example, the metric can be an F1-score, AUCPR, or geometric mean. In certain non-limiting embodiments, the decision threshold of 0.9 corresponds to the maximum F1-score, with the averages F1 score being 0.6825. In other embodiments, however, a decision threshold of 0.2, 0.3, 0.4 0.5, 0.6 , 0.7, or 0.8 can be used.

### Example 3

In certain non-limiting embodiments, prediction model can include an RNN. The RNN include a 5-3-3 architecture, 10-fold, and 18 epochs. The RNN would evaluate six selected features, such as one or more biomarkers and demographic information. For example, the six selected features can be BUN, urine sg, visiting age, creatinine, urine protein, and/or weight. As compared to Example 2, Example 3 does not use amylase in the prediction model. Amylase was removed because cholesterol has a small or non-existent impact on the prediction model. Feature selection conducted using top-down wrapper and bottom-up wrapper supported the removal of Amylase from the prediction model in Example 3. By removing amylase, the prediction model of Example 3 relies on six rather than seven features. Doing so can decrease the performance of the prediction model by an F1 score of 0.2%. Overall, however, the prediction model in Example 3 achieved a 79.1% sensitivity, 93.8% specificity, 94.2% AUCROC, 91.6% AUCPR, and 82.4% F1 score.

Amylase can be a calcium-dependent enzyme which hydrolyzes complex carbohydrates at alpha 1,4-linkages to form maltose and glucose. Amylase can be filtered by renal tubules and resorbed (inactivated) by tubular epithelium. Active enzyme does not appear in urine. Small amounts of amylase can be taken up by Kupffer cells in the liver. In healthy dogs, for example, 14% of amylase can be bound to globulins. Because of this polymerization, canine amylase can have variable (high) molecular weights and cannot be normally filtered by the kidney. In dogs with renal disease, this polymerized (macroamylase) amylase can be found in higher concentration (from 5-62% of total amylase activity) and can contribute to the hyperamylasemia seen in CKD.

In some non-limiting embodiments, the performance of the prediction model in Example 3 was boosted to achieve an 84.1% sensitivity, 94.2% specificity, 95.6% AUCROC, 93.8% AUCPR, and 85.8% F1 score. The performance improvement was achieved by modifying the training of the prediction model. In particular, after sub-sampling one or more bootstrapped sample with different seeds can be formed, and the training subset can be selected using RNN cross-validation predictor. The training subset yielding the best F1 score can be selected. For example, an RNN with 5-3-3 configuration or architecture, with 10 folds, and 8 epochs can be selected.

In certain non-limiting embodiments, a decision threshold of 0.5 can be used to optimize the true positive rate (e.g., sensitivity or recall) against the false positive rate (e.g., specificity). In some non-limiting embodiments, the chosen temperature parameter T was found equal to 1.296 by minimizing the negative log likelihood using a quasi-newton Broyden-Fletcher-Goldfarb-Shanno (BFGS) optimizer.

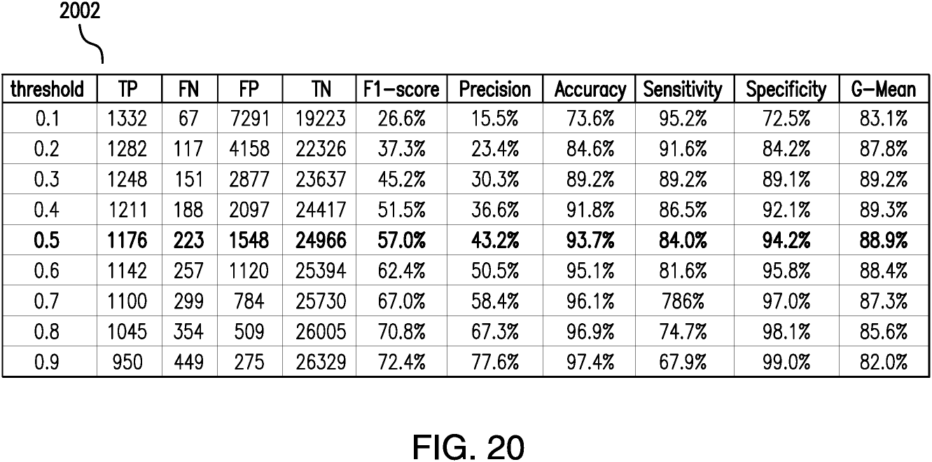

FIG. 20 illustrates a decision threshold table 2002 according to certain embodiments described herein. For example, the table shows a decision threshold ranging from 0.1 to 0.9 with step 0.1. The G-mean shown in FIG. 13 can indicate the geometric mean of sensitivity and specificity. The decision threshold can be chosen based on one of more of the following: F1 score, precision, accuracy, sensitivity, specificity, and G-mean.

Sensitivity can be the percentage of true positives with the disease. A highly sensitive test can be useful for ruling out a disease with a negative test but not necessarily ruling in the disease. On the other hand, specificity can be the percentage of true negatives without disease and can be useful for ruling in a positive test (if high specificity) but not ruling out a disease. In the setting of a highly sensitive and specific test, while sensitivity is easily understood (if you do not have the test positive, then the disease may not be present), specificity leads to confusion because, rather than being focused on having the disease, the focus is on not having the disease. Highly specific tests can have low false positive rates and highly sensitive tests can have low false negative rates. Sensitivity and specificity can be on a continuum with an inverse relationship where perfect sensitivity (close to 100%) will lead to a loss in specificity and vice-versa. The receiver operator curve (ROC) can be the statistical and graphical description of the process showing the equilibrium between sensitivity and specificity. A similar continuum can be found when describing sensitivity and specificity of any classification and/or diagnostic criteria.

Positive predictive value (PPV) can illustrate the above point. PPV can be the proportion of true positives to the number of positive tests and can be a measure of the accuracy or performance of a diagnostic test. Negative predictive value (NPV) can be the opposite, a proportion of the number of true negatives to the number of negative tests. Both PPV and NPV can be highly dependent on the prevalence of CKD.

Although the presently disclosed subject matter and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

1. A computer system for identifying susceptibility of a dog to develop chronic kidney disease (CKD), the computer system comprising:
a processor; and
a memory that stores code that, when executed by the processor, causes the computer system to:
(a) receive at least one of:
(i) one or more biomarkers of the dog, wherein the one or more biomarkers comprises information relating to at least one of a urine specific gravity, a creatinine, a urine protein, a blood urea nitrogen (BUN); or
(ii) demographic information of the dog, wherein the demographic information includes at least one of age or weight of the dog;
(b) process at least one of the one or more biomarkers or demographic information of the dog using a prediction model, wherein the prediction model comprises a recurrent neural network; and
(c) determine a probability risk score of the dog for developing CKD based on the processed one or more biomarkers or demographic information.

2. The computer system according to claim 1, wherein the computer system is caused to:
determine a customized recommendation based on the probability risk of the dog for developing CKD.

3. The computer system according to claim 1, wherein the computer system is caused to:
transmit the customized recommendation to a user equipment of a veterinarian, owner, or caregiver of the dog.

4. The computer system according to claim 2, wherein the customized recommendation comprises at least one of:
(a) one or more therapeutic interventions;
(b) one or more dietary recommendations;
(c) one or more renal sparing strategies; or
(d) one or more tests for disease progression.

5. The computer system according to claim 4, wherein:
(i) the one or more renal sparing strategies comprise avoidance of non-steroidal antiinflammatories, aminoglycosides, or any combination thereof; and/or
(ii) the one or more tests for disease progression comprise testing of serum parathyroid hormone levels.

6. The computer system according to claim 1, wherein the recurrent neural network comprises a hidden layer architecture with three layers, the three layers comprising a first layer with five nodes, a second layer with three nodes, and a third layer with three nodes.

7. The computer system according to claim 1, wherein the recurrent neural network undergoes a ten-fold cross-validation process and is trained over eight or eighteen epochs.

8. The computer system according to claim 1, wherein the one or more biomarker comprises information relating to an amylase.

9. The computer system according to claim 1, wherein the recurrent neural network is trained using a training dataset, wherein the training dataset comprises the one or more biomarkers and the demographic information for a plurality of other dogs.

10. The computer system according to claim 1, wherein the prediction model further comprises the recurrent neural network with long short-term memory (LSTM).

11. The computer system according to claim 1 wherein the decision threshold for developing the CKD using the recurrent neural network is about 0 to about 1.

12. The computer system according to claim 1 wherein the decision threshold for developing the CKD using the recurrent neural network is about 0.5.

13. The computer system according to claim 1, wherein the compute system is caused to:
impute one or more missing values from the one or more biomarkers of the dog or the demographic information of the dog.

14. The computer system according to claim 13, wherein the imputation is a linear regression.

15. The computer system according to claim 13, wherein the imputation is based on an age of the dog.

16. The compute system according to claim 13, wherein the imputation is based on the number of missing values.

17. A method for identifying susceptibility of a dog to develop chronic kidney disease (CKD), the method comprising:
(a) receiving at least one of:
(i) one or more biomarkers of the dog, wherein the one or more biomarkers comprises information relating to at least one of a urine specific gravity, a creatinine, a urine protein, a blood urea nitrogen (BUN); or
(ii) demographic information of the dog, wherein the demographic information includes at least one of age or weight of the dog;
(b) processing at least one of the one or more biomarkers or demographic information of the dog using a prediction model, wherein the prediction model comprises a recurrent neural network; and
(c) determining a probability risk score of the dog for developing CKD based on the processed one or more biomarkers or demographic information.

18. The method according to claim 17, further comprising:
determining a customized recommendation based on the probability risk of the dog for developing CKD.

19. The method according to claim 17, further comprising:
transmitting the customized recommendation to a user equipment of a veterinarian, owner, or caregiver of the dog.

20. The method according to claim 18, wherein the customized recommendation comprises at least one of:
(a) one or more therapeutic interventions;
(b) one or more dietary recommendations;
(c) one or more renal sparing strategies; or
(d) one or more tests for disease progression.

21. The method according to claim 20, wherein:
(i) the one or more renal sparing strategies comprise avoidance of non-steroidal antiinflammatories, aminoglycosides, or any combination thereof; and/or
(ii) the one or more tests for disease progression comprise testing of serum parathyroid hormone levels.

22. The method according to claim 17, wherein the recurrent neural network comprises a hidden layer architecture with three layers, the three layers comprising a first layer with five nodes, a second layer with three nodes, and a third layer with three nodes.

23. The method according to claim 17, wherein the recurrent neural network undergoes a ten-fold cross-validation process and is trained over eight or eighteen epochs.

24. The method according to claim 17, wherein the one or more biomarker comprises information relating to an amylase.

25. The method according to claim 17, wherein the recurrent neural network is trained using a training dataset, wherein the training dataset comprises the one or more biomarkers and the demographic information for a plurality of other dogs.

26. The method according to claim 17, wherein the prediction model further comprises the recurrent neural network with long short-term memory (LSTM).

27. The method according to claim 17 wherein the decision threshold for developing the CKD using the recurrent neural network is about 0.0 to about 1.0.

28. The method according to claim 17, wherein the decision threshold for developing the CKD using the recurrent neural network is about 0.5.

29. The method according to claim 17, further comprising:
imputing one or more missing values from the one or more biomarkers of the dog or the demographic information of the dog.

30. The method according to claim 17, wherein the imputation is a linear regression.

31. The method according to claim 17-29, wherein the imputation is based on an age of the dog.

32. The method according to claim 17 wherein the imputation is based on the number of missing values.

33-62. (canceled)
